(19) United States
(12) Patent Application Publication
(10) Pub. No.: US 2014/0005181 A1
(43) Pub. Date: Jan. 2, 2014

(54) SMALL MOLECULE ANTAGONISTS OF THE APELIN RECEPTOR FOR THE TREATMENT OF DISEASE

(71) Applicant: Sanford-Burnham Medical Research Institute, La Jolla, CA (US)

(72) Inventors: Layton Smith, Orlando, FL (US); Anthony B. Pinkerton, San Diego, CA (US)

(73) Assignee: Sanford-Burnham Medical Research Institute, La Jolla, CA (US)

(21) Appl. No.: 13/923,131

(22) Filed: Jun. 20, 2013

Related U.S. Application Data
(60) Provisional application No. 61/662,641, filed on Jun. 21, 2012.

(51) Int. Cl. C07D 309/36; C07D 413/14; C07D 409/14; C07D 405/14; C07D 405/12; C07D 417/14

(52) U.S. Cl. CPC C07D 309/36; C07D 405/12; C07D 417/14; C07D 409/14; C07D 405/14; C07D 413/14
USPC 514/227.8; 514/274; 544/316; 514/459; 549/417; 514/231.5; 544/149; 544/58.7; 514/414; 548/465; 514/326; 546/207; 435/375; 544/318; 544/374; 514/254.1

(57) ABSTRACT

The present disclosure relates to compounds and methods for treating a disease mediated by apelin.

### CROSS-REFERENCE TO RELATED APPLICATIONS

The present application claims the benefit under 35 U.S.C. §119(e) to U.S. Provisional Application Ser. No. 61/662,641, filed Jun. 21, 2012, the contents of which is hereby incorporated by reference in its entirety.

### STATEMENT OF GOVERNMENT-SPONSORED RESEARCH

This invention was made with United States government support awarded by the following agencies: National Institutes of Health under Grant No. 1R21NS059422-01, and National Institutes of Health Molecular Libraries under Grant No. U54 HG005033-03. The United States government has certain rights in the invention.

### FIELD OF THE DISCLOSURE

The present disclosure relates to compounds and methods for treating a disease mediated by apelin.

### BACKGROUND OF THE DISCLOSURE

Apelin is a circulating peptide hormone, synthesized and secreted by a number of cell types including those of the cardiovascular, endocrine, gastrointestinal and nervous systems. Apelin was recently identified as the endogenous ligand of the APJ, a formerly orphaned G-protein coupled receptor (GPCR) with a similarly broad distribution of expression (ref 1). APJ is also referred to as APJ receptor or apelin receptor.

The tissue distribution of both apelin and APJ suggests an involvement of this system in a range of physiological functions. Indeed, apelin has been shown to play a role in mediating gastrointestinal function (ref 2-6), food and water consumption (ref 7-11), energy metabolism (ref 12-16), and cardiovascular homeostasis (ref 17-21). In addition to normal physiological function, apelin has been associated with the pathogenesis of cardiovascular and metabolic diseases including atherosclerosis (ref 22-23), hypertension (ref 24-26), heart failure (ref 27-28) and both type 1 (ref 29) and type 2 diabetes mellitus (ref 30-31). Despite this abundance of work, several unanswered questions regarding the role apelin and APJ in normal physiology and pathology remain. Small molecule probes of the apelin/APJ system would advance apelin research significantly. In particular an APJ antagonist would be a useful tool for determining the function and pharmacology of APJ, and ultimately to validate the importance of this system in animal models.

### SUMMARY OF THE DISCLOSURE

Provided herein are small molecule antagonists of APJ.

In one aspect, the present disclosure provides a method of inhibiting the activity of APJ or apelin signaling in a cell, comprising contacting the cell with an effective amount of a compound as described herein. Such contacting can be in vitro or in vivo. While in vivo, the contacting can be achieved by administering the compound to a subject, such as a mammalian subject in particular a human subject, leading to contact between the compound and the cell.

In one aspect, the present disclosure provides a method of treating a disease mediated by apelin in a patient in need thereof, comprising administering to the patient a therapeutically effective amount of a compound as described herein.

In another embodiment, provided is a method of treating a tumor or disease caused by abnormal angiogenesis in a patient in need thereof, comprising administrating to the patient a therapeutically effective amount of a compound as described herein.

In one embodiment the compound is of formula (I):

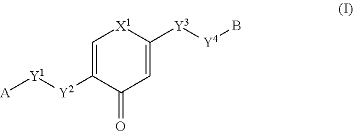

wherein:
X¹ is selected from the group consisting of —O— and —N(R⁵)—;
A is selected from the group consisting of alkyl, phenyl and 5- or 6-membered heteroaryl, wherein each phenyl and heteroaryl is independently optionally substituted with from one to three R²⁰;
B is selected from the group consisting of phenyl and 5- or 6-membered heteroaryl, wherein each phenyl and heteroaryl is independently optionally substituted with from one to three R²⁰;
Y¹ and Y² are each independently selected from the group consisting of a bond, —C(O)O—, —C(O)—, —CH₂O—, —N(R¹)—, —C(R²)(R³)— and —S(O)₂—;
Y³ and Y⁴ are each independently selected from the group consisting of a bond, —S—, —O—, —N(R¹)—, —C(R²)(R³)—, —C(O)— and —S(O)₂—;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof.

Another aspect of the present disclosure provides a compound of formula (II):

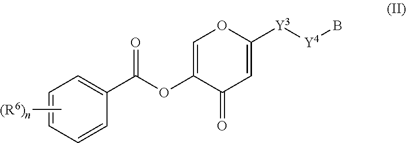

wherein:
B is selected from the group consisting of phenyl, heterocycloalkyl, and 5- or 6-membered heteroaryl, wherein each phenyl, heterocycloalkyl, and heteroaryl is independently optionally substituted with from one to three R²⁰;
Y³ and Y⁴ are each independently selected from the group consisting of a bond, —S—, —O—, —N(R¹)—, —C(R²)(R³)—, —C(O)— and —S(O)₂—;
n is 1, 2, or 3;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R⁶ is independently selected from the group consisting of halogen, —CN, —NO₂, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof;
provided that:
   (A) when Y³ is —CH₂—; Y⁴ is —S—; and B is unsubstituted pyrimidin-2-yl; then A is not phenyl, 3-NO₂-4-Me-phenyl, 3-NO₂-phenyl, 4-Cl-phenyl, 4-NO₂-phenyl, 4-CF₃-phenyl, 4-Br-phenyl, 4-Me-phenyl, 4-OMe-phenyl, 4-OEt-phenyl, 4-OiPr-phenyl, 4-OBu-phenyl, 4-tBu-phenyl, 4-F-phenyl, 4-SO₂NMe₂-phenyl, 4-SO₂N(CH₂)₄-phenyl, 3,4-diMe-phenyl, 2-NO₂-5-Cl-phenyl, 2-Cl-5-NO₂-phenyl, 2-Me-3-NO₂-phenyl, 4-Ph-phenyl, 4-SO₂NEt₂-phenyl, 4-SO₂N(CH₂)₅-phenyl, 4-SO₂-morpholine-phenyl, or 2-NO₂-4,5-diOMe-phenyl;
   (B) when Y³ is —CH₂—; Y⁴ is —S—; and B is 4-methyl-pyrimidin-2-yl; then A is not 3-NO₂-4-Cl-phenyl, 4-SO₂N(CH₂)₄-phenyl, 2-NO₂-5-Cl-phenyl, or 2-Cl-5-NO₂-phenyl; and
   (C) when Y³ is —CH₂—; Y⁴ is —S—; and B is 4,6-dimethyl-pyrimidin-2-yl; then A is not 3-NO₂-4-Cl-phenyl, 3-NO₂-4-Me-phenyl, 4-SO₂NMe₂-phenyl, 2-NO₂-5-Cl-phenyl, 2-Cl-5-NO₂-phenyl, or 2-Me-3-NO₂-phenyl.

### BRIEF DESCRIPTION OF THE FIGURES

FIG. 1 shows a representative dose response curve for Compound 6. The compound antagonized Ap13-mediated activation of APJ in a concentration-dependent manner in both a cAMP assay (▪), and a β-arrestin recruitment assay (•). Data plotted are the mean±SEM % inhibition of Ap13. Curves represent the best fit of a four parameter logistic generated using GraphPad Prism5.

FIGS. 2 and 3 show the data against a GPCR binding assay panel for Compound 6.

FIG. 4 shows a representative dose response curve for Compound 6 in the cAMP assay.

FIG. 5 shows a representative dose response curve for Compound 6 in the APJ receptor internalization assay.

FIG. 6 shows a representative dose response curve for Compound 6 in the DiscoveRx assay for apelin receptor (APJ) antagonist activity.

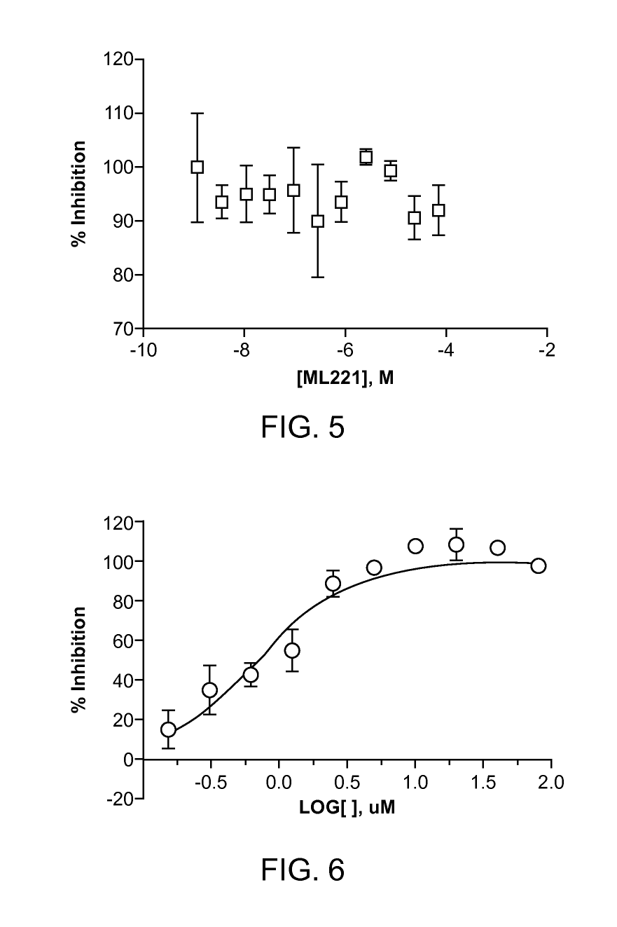

### DETAILED DESCRIPTION

### Definitions

As used herein, unless otherwise stated, the singular forms “a,” “an,” and “the” include plural reference. Thus, for example, a reference to “a lipid” includes a plurality of lipid molecules.

As used herein, the term “treating” refers to the medical management of a subject with the intent that a cure, amelioration, or prevention of the disease, pathological condition, or disorder or a related or accompanying disorder will result. This term includes active treatment, that is, treatment directed specifically toward improvement of the disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease: preventive treatment, that is, treatment directed to prevention of the disease; and supportive treatment, that is, treatment employed to supplement another specific therapy directed toward the improvement of the disease. The term “treating” also includes symptomatic treatment, that is, treatment directed toward constitutional symptoms of the disease.

As used herein, “patient” refers to a mammal (e.g., human, dog, cat, and horse) that is suffering from a disease. In some aspects, the disease is mediated by apelin or APJ.

By “a therapeutically effective amount” is meant the amount of a compound, alone or in combination with another therapeutic regimen, required to treat or prevent a disease mediated by apelin in a clinically relevant manner. A sufficient amount of an active compound used to practice the present disclosure for therapeutic treatment of a disease mediated by apelin varies depending upon the manner of administration, the age, body weight, and general health of the subject. Such amounts are determined by the skilled clinician.

The terms “treating” or “treatment” in reference to a particular disease includes ameliorating the symptom or underlying conditions or a disease and prevention of the disease.

The terms “administration of” and or “administering a” compound should be understood to mean providing a compound of the disclosure or pharmaceutical composition to the subject in need of treatment.

As used herein, the term “alkyl” refers to a straight or branched hydrocarbon radical, which may be fully saturated, mono- or polyunsaturated having from 1 to 20, or from 1 to 8, or from 1 to 6 or from 1 to 4 carbon atoms. Examples include, but are not limited to, groups such as methyl, ethyl, n-propyl, isopropyl, n-butyl, t-butyl, isobutyl, sec-butyl, n-pentyl, n-hexyl, n-heptyl, n-octyl, and the like.

As used herein, the term “cycloalkyl” refers to a cyclic hydrocarbon radical, which may contain one or more fused rings (i.e., heterocycloalkyl aryl or heteroaryl), so long as the ring which is directly attached to the remainder of the molecule is a cycloalkyl. Examples of cycloalkyl include, but are not limited to, cyclopropyl, cyclobutyl, cyclopentyl, cyclohexyl, 1-cyclohexenyl, 3-cyclohexenyl, cycloheptyl, and the like.

As used herein, the term “heterocycloalkyl” refers to a cycloalkyl group as defined herein, containing one or more (e.g., from one to three) heteroatom(s). Examples of heterocycloalkyl groups include, but are not limited to, 1-(1,2,5,6-tetrahydropyridyl), 1-piperidinyl, 2-piperidinyl, 3-piperidinyl, 4-morpholinyl, 4-thiomorpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A heterocycloalkyl group can be attached to the remainder of the molecule through a carbon or heteroatom.

As used herein, the term “alkoxy” refers to the moiety —O-alkyl, wherein alkyl is as defined above. Examples of alkoxy structures that are within the purview of the definition include, but are not limited to, methoxy, ethoxy, propoxy, isopropoxy, butoxy, iso-butoxy, sec-butoxy, tert-butoxy, pentoxy, 3-pentoxy, and hexyloxy.

As used herein, the term “haloalkyl” refers to an alkyl group as defined above containing one or more (e.g., from one to three) halogen substituents, and is mean to include, but not be limited to, trifluoromethyl, 2,2,2-trifluoroethyl, 4-chlorobutyl, 3-bromopropyl, and the like.

As used herein, the term “haloalkoxy” refers to an alkoxy group, as defined above, containing one or more (e.g., from one to three) halogen substituents.

As used herein, the term “halogen” refers to a fluorine, chlorine, bromine, or iodine atom.

As used herein, the term “heteroaryl” refers to an aromatic substituent which can be a single ring, or multiple fused rings, containing from four to eight carbon atoms and from one to four heteroatoms selected from N, O, and S, wherein any nitrogen or sulfur atom may be oxidized. Non-limiting examples of heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Where a heteroalkyl, heterocycloalkyl, or heteroaryl includes a specific number of members (e.g., “5 or 6 membered”), the term “member” referrers to a carbon or heteroatom.

The term “pharmaceutically acceptable salt” refers to salts that may be used where the compounds used in the methods of the disclosure are sufficiently basic or acidic to form stable, nontoxic acid or base salts. These salts may be prepared by methods known to those skilled in art. Examples of pharmaceutically acceptable salts include organic acid addition salts formed with acids which form a physiological acceptable anion, for example, oxalate, tosylate, methanesulfonate, acetate, citrate, malonate, tartarate, succinate, benzoate, ascorbate, ketoglutarate, and glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example by treating a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the disclosure can exist in unsolvated forms as well as solvated and/or hydrated forms. In general, the solvated and unsolvated forms are encompassed within the scope of the disclosure. Certain compounds of the disclosure may exist in one or more crystalline or amorphous forms. In general, all physical forms are intended to be within the scope of the disclosure.

As used herein, the term “polymorph” refers to one or more crystalline forms of the compound. Any particular polymorph can exhibit packing polymorphism (difference in crystal packing), conformational polymorphism (different conformers of the same molecule), or pseudopolymorphism or solvomorphism (different crystal types are the result of hydration or solvation).

Certain compounds of the disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids. The disclosure is meant to include compounds in racemic and optically pure forms. Optically active (R)- and (S)-, or (D)- and (L)-isomers may be prepared using chiral synthons or chiral reagents, or resolved using conventional techniques. Thus, the structures depicted herein are also meant to include all stereochemical forms of the structure. All single stereochemical isomers as well as enantiomeric and diastereomeric mixtures of the present compounds are within the scope of the disclosure. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another. It will be apparent to one skilled in the art that certain compounds of the disclosure may exist in tautomeric forms. All such tautomeric forms of the compounds being within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or the replacement of a ¹²C-carbon by ¹³C-enriched carbon are within the scope of the disclosure.

The compounds of the disclosure may also contain unnatural proportions of atomic isotopes at one or more of atoms that constitute such compounds. For example, the compounds may be radiolabeled with radioactive isotopes, such as for example tritium (³H), iodine-125 (²⁵I) or carbon-14 (¹⁴C). All isotopic variations of the compounds of the disclosure, whether radioactive or not, are encompassed within the scope of the disclosure.

The term “pharmaceutically acceptable salts” is meant to include salts of active compounds which are prepared with relatively nontoxic acids or bases, depending on the particular substituent moieties found on the compounds described herein. When compounds of the disclosure contain relatively acidic functionalities, base addition salts can be obtained by contacting the neutral form of such compounds with a sufficient amount of the desired base, either neat or in a suitable solvent. Examples of pharmaceutically acceptable base addition salts include the sodium, potassium, calcium, ammonium, organic amino, or magnesium salt. When compounds of the disclosure contain relatively basic functionalities, acid addition salts can be obtained by contacting the neutral form with a sufficient amount of the desired acid, either neat or in a suitable solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or the like, as well as the salts derived from organic acids, such as acetic, propionic, isobutyric, maleic, malonic, benzoic, succinic, suberic, fumaric, lactic, mandelic, phthalic, benzenesulfonic, p-tolylsulfonic, citric, tartaric, methanesulfonic acid, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19).

Description of compounds of the disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

By “pharmaceutically acceptable” it is meant the carrier, diluent or excipient must be compatible with the other ingredients of the formulation and not deleterious to the recipient thereof.

### Compounds

Provided herein are compounds of Formula (I):

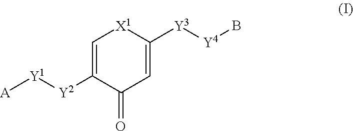

wherein:
X¹ is selected from the group consisting of —O— and —N(R¹)—;
A is selected from the group consisting of alkyl, phenyl and 5- or 6-membered heteroaryl, wherein each phenyl and heteroaryl is independently optionally substituted with from one to three R²⁰;
B is selected from the group consisting of phenyl, heterocycloalkyl, and 5- or 6-membered heteroaryl, wherein each phenyl, heterocycloalkyl, and heteroaryl is independently optionally substituted with from one to three R²⁰;
Y¹ and Y² are each independently selected from the group consisting of a bond, —C(O)O—, —C(O)—, —CH₂O—, —N(R¹)—, —C(R²)(R³)— and —S(O)₂—;
Y³ and Y⁴ are each independently selected from the group consisting of a bond, —S—, —O—, —N(R¹)—, —C(R²)(R³)—, —C(O)— and —S(O)₂—;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof;
provided that:
   (A) when X¹ is O; Y¹ is a bond; Y² is —C(O)O—; Y³ is —CH₂—; Y⁴ is —S—; and B is unsubstituted pyrimidin-2-yl; then A is not phenyl, 3-NO₂-4-Me-phenyl, 3-NO₂-phenyl, 4-Cl-phenyl, 4-NO₂-phenyl, 4-CF₃-phenyl, 4-Br-phenyl, 4-Me-phenyl, 4-OMe-phenyl, 4-OEt-phenyl, 4-OiPr-phenyl, 4-OBu-phenyl, 4-tBu-phenyl, 4-F-phenyl, 4-SO₂NMe₂-phenyl, 4-SO₂N(CH₂)₄-phenyl, 3,4-diMe-phenyl, 2-NO₂-5-Cl-phenyl, 2-Cl-5-NO₂-phenyl, 2-Me-3-NO₂-phenyl, 4-Ph-phenyl, 4-SO₂NEt₂-phenyl, 4-SO₂N(CH₂)₅-phenyl, 4-SO₂-morpholine-phenyl, or 2-NO₂-4,5-diOMe-phenyl;
   (B) when X¹ is O; Y¹ is a bond; Y² is —C(O)O—; Y³ is —CH₂—; Y⁴ is —S—; and B is 4-methyl-pyrimidin-2-yl; then A is not 3-NO₂-4-Cl-phenyl, 4-SO₂N(CH₂)₄-phenyl, 2-NO₂-5-Cl-phenyl, or 2-Cl-5-NO₂-phenyl; and
   (C) when X¹ is O; Y¹ is a bond; Y² is —C(O)O—; Y³ is —CH₂—; Y⁴ is —S—; and B is 4,6-dimethyl-pyrimidin-2-yl; then A is not 3-NO₂-4-Cl-phenyl, 3-NO₂-4-Me-phenyl, 4-SO₂NMe₂-phenyl, 2-NO₂-5-Cl-phenyl, 2-Cl-5-NO₂-phenyl, or 2-Me-3-NO₂-phenyl.

In certain embodiments, A is selected from the group consisting of phenyl and 5- or 6-membered heteroaryl, wherein each phenyl and heteroaryl is independently optionally substituted with from one to three R²⁰.

In certain embodiments, A is optionally substituted phenyl and B is optionally substituted heteroaryl.

In certain embodiments, X¹ is O. In other embodiments, X¹ is NH₂.

In certain embodiments, —Y¹—Y²— is —C(O)O—.

In one embodiment, provided is a compound of formula (II):

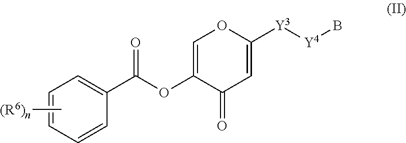

wherein:
B is selected from the group consisting of phenyl, heterocycloalkyl, and 5- or 6-membered heteroaryl, wherein each phenyl, heterocycloalkyl, and heteroaryl is independently optionally substituted with from one to three R²⁰;
Y³ and Y⁴ are each independently selected from the group consisting of a bond, —S—, —O—, —N(R¹)—, —C(R²)(R³)—, —C(O)— and —S(O)₂—;
n is 1, 2, or 3;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R⁶ is independently selected from the group consisting of halogen, —CN, —NO₂, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof;
provided that:
   (A) when Y³ is —CH₂—; Y⁴ is —S—; and B is unsubstituted pyrimidin-2-yl; then A is not phenyl, 3-NO₂-4-Me-phenyl, 3-NO₂-phenyl, 4-Cl-phenyl, 4-NO₂-phenyl, 4-CF₃-phenyl, 4-Br-phenyl, 4-Me-phenyl, 4-OMe-phenyl, 4-OEt-phenyl, 4-OiPr-phenyl, 4-OBu-phenyl, 4-tBu-phenyl, 4-F-phenyl, 4-SO₂NMe₂-phenyl, 4-SO₂N(CH₂)₄-phenyl, 3,4-diMe-phenyl, 2-NO₂-5-Cl-phenyl, 2-Cl-5-NO₂-phenyl, 2-Me-3-NO₂-phenyl, 4-Ph-phenyl, 4-SO₂NEt₂-phenyl, 4-SO₂N(CH₂)₅-phenyl, 4-SO₂-morpholine-phenyl, or 2-NO₂-4,5-diOMe-phenyl;
   (B) when Y³ is —CH₂—; Y⁴ is —S—; and B is 4-methyl-pyrimidin-2-yl; then A is not 3-NO₂-4-Cl-phenyl, 4-SO₂N(CH₂)₄-phenyl, 2-NO₂-5-Cl-phenyl, or 2-Cl-5-NO₂-phenyl; and
   (C) when Y³ is —CH₂—; Y⁴ is —S—; and B is 4,6-dimethyl-pyrimidin-2-yl; then A is not 3-NO₂-4-Cl-phenyl, 3-NO₂-4-Me-phenyl, 4-SO₂NMe₂-phenyl, 2-NO₂-5-Cl-phenyl, 2-Cl-5-NO₂-phenyl, or 2-Me-3-NO₂-phenyl.

In certain embodiments, —Y³—Y⁴— is —CH₂—, —CH₂—N(CH₂—C₆H₅)—, —CH₂—S—, or —CH₂—S(O)₂—.

In certain embodiments, B is selected from the group consisting of phenyl, pyrimidinyl, morpholinyl, thiomorpholinyl, and piperazinyl, wherein each phenyl, pyrimidinyl, morpholinyl, thiomorpholinyl, and piperazinyl is optionally substituted with from one to three R²⁰.

In certain embodiments, each R²⁰ is independently selected from the group consisting of halogen, —NO₂, —CN, —S(O)₂—N(CH₃)₂, —S(O)₂-heterocycloalkyl, alkyl, alkoxy, haloalkyl, haloalkoxy, phenyl, and heteroaryl.

In certain embodiments, provided is a compound of formula (III):

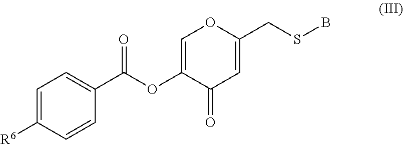

wherein:
B is selected from the group consisting of phenyl, heterocycloalkyl, and 5- or 6-membered heteroaryl, wherein each phenyl, heterocycloalkyl, and heteroaryl is independently optionally substituted with from one to three R²⁰;
R⁶ is selected from the group consisting of halogen, —CN, —NO₂, alkyl, cycloalkyl, heterocycloalkyl, alkoxy, haloalkyl, haloalkoxy, and 5- or 6-membered heteroaryl; and
each R²⁰ is independently selected from the group consisting of halogen, oxo, —C(O)-phenyl, alkyl, alkoxy, haloalkoxy, and phenyl;
or a pharmaceutically acceptable salt, polymorph, solvate, tautomer, or N-oxide thereof;
provided that when B is unsubstituted pyrimidin-2-yl; then R⁶ is not —F, —Cl, —Br, —NO₂, —CF₃, -Me, —OMe, —OEt, -OiPr, —OBu, or -tBu.

In certain embodiments, B is selected from the group consisting of:

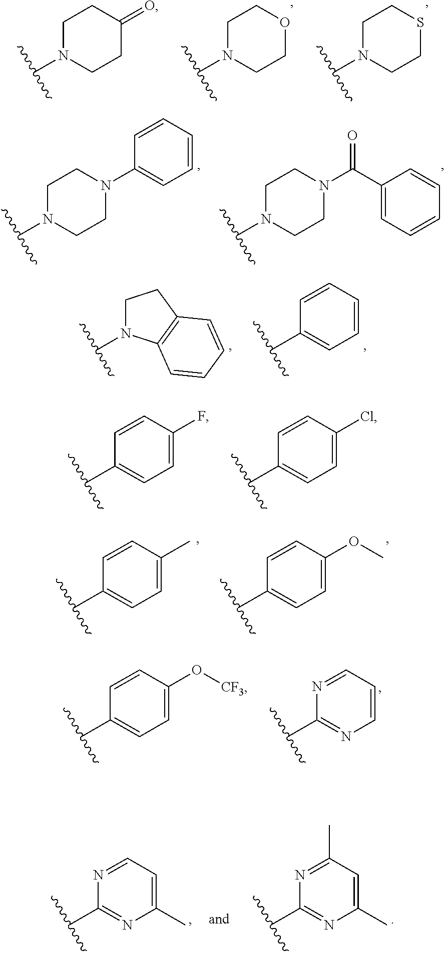

In certain embodiments, R⁶ is selected from the group consisting of bromo, —NO₂, —CN, or trifluoromethyl.

In certain embodiments, provided is a compound selected from the group consisting of:

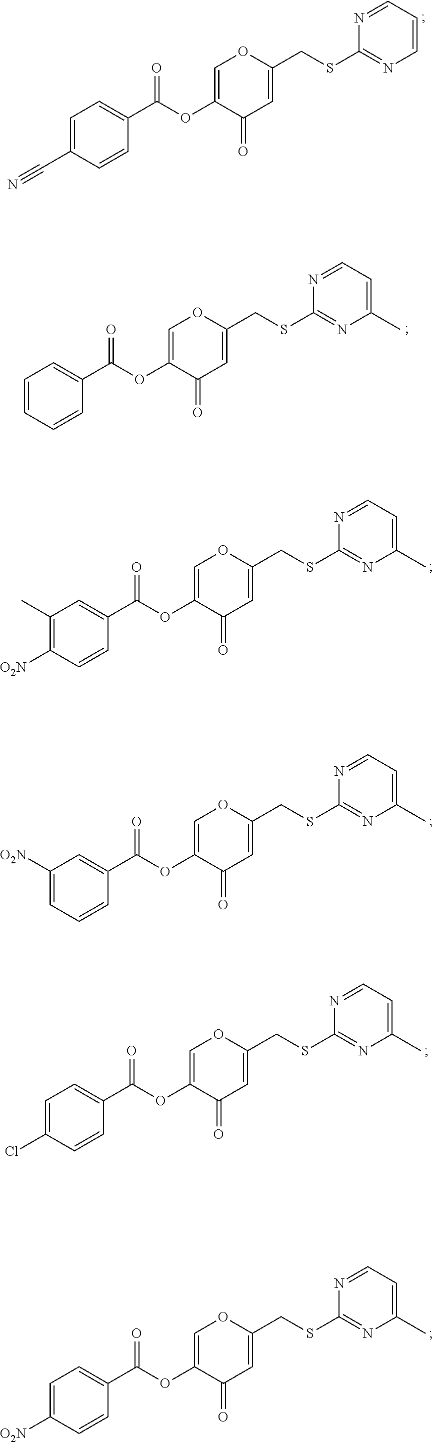

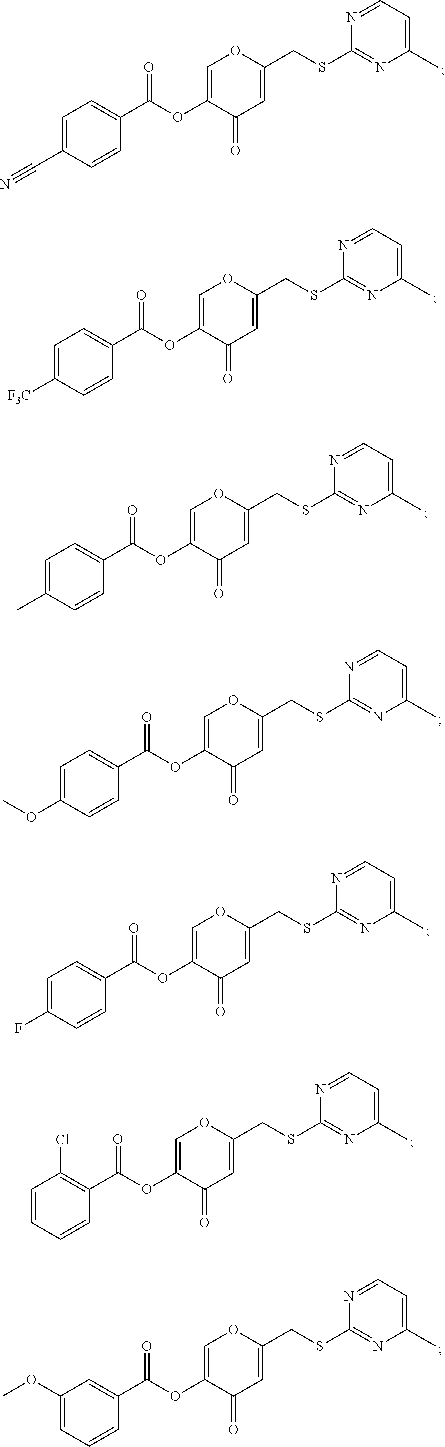

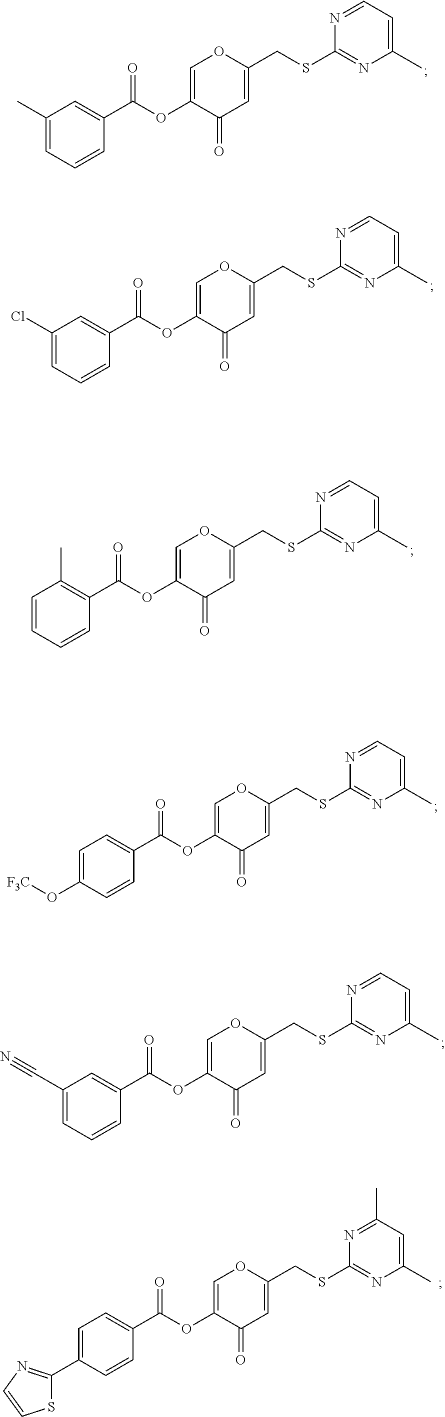

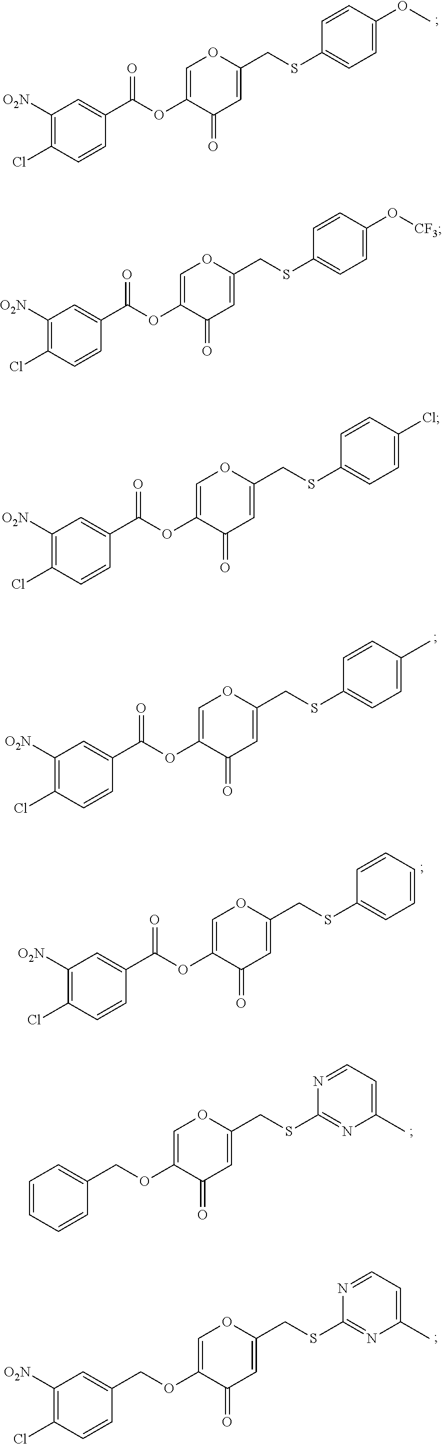

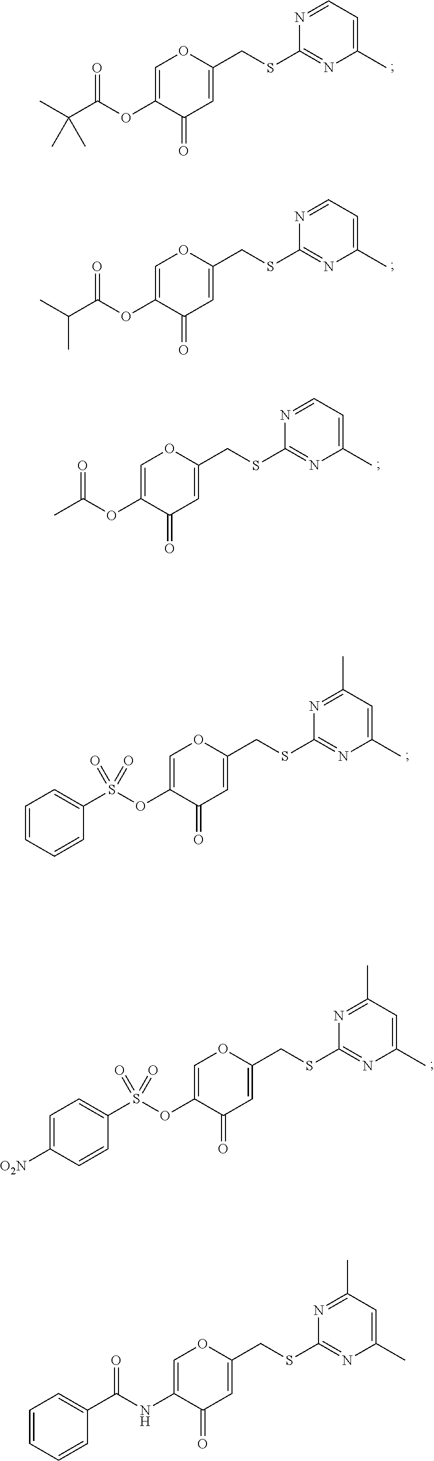

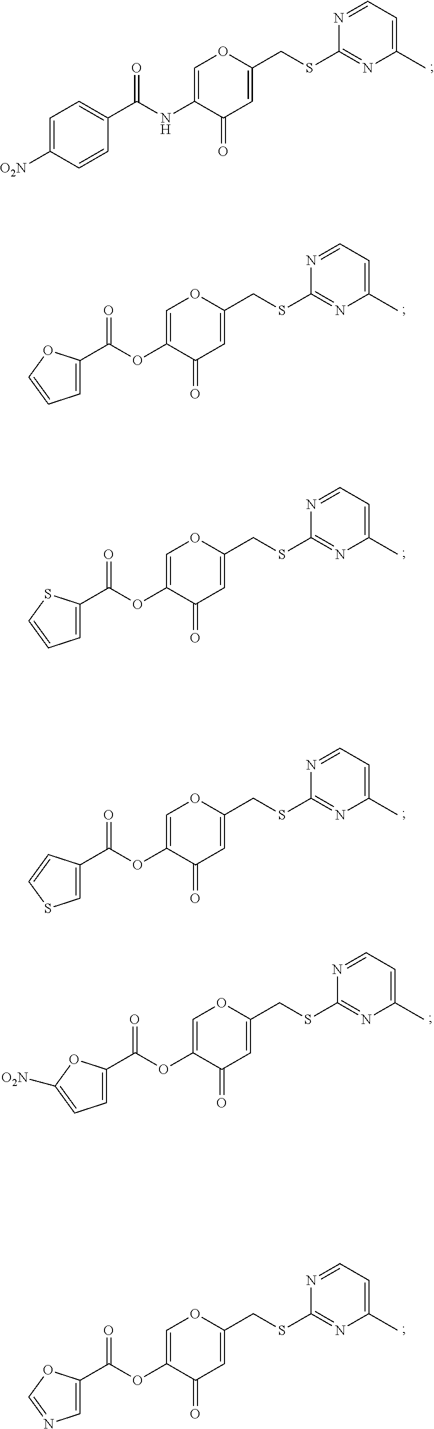 [SMILES: Cc1ccnc(SCc2cc(=O)c(NC(=O)c3ccc([N+](=O)[O-])cc3)co2)n1 ; Cc1ccnc(SCc2cc(=O)c(OC(=O)c3ccco3)co2)n1 ; Cc1ccnc(SCc2cc(=O)c(OC(=O)c3cccs3)co2)n1 ; Cc1ccnc(SCc2cc(=O)c(OC(=O)c3ccsc3)co2)n1 ; Cc1ccnc(SCc2cc(=O)c(OC(=O)c3ccc([N+](=O)[O-])o3)co2)n1 ; Cc1ccnc(SCc2cc(=O)c(OC(=O)c3cnco3)co2)n1]

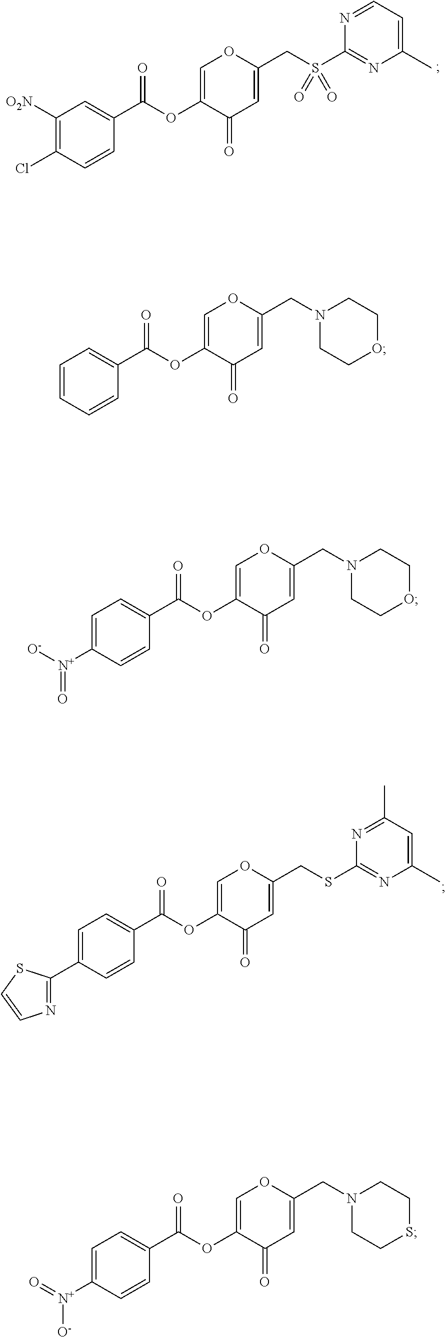 [SMILES: Cc1ccnc(S(=O)(=O)Cc2cc(=O)c(OC(=O)c3ccc(Cl)c([N+](=O)[O-])c3)co2)n1 ; O=C(Oc1coc(CN2CCOCC2)cc1=O)c1ccccc1 ; O=C(Oc1coc(CN2CCOCC2)cc1=O)c1ccc([N+](=O)[O-])cc1 ; Cc1cc(C)nc(SCc2cc(=O)c(OC(=O)c3ccc(-c4nccs4)cc3)co2)n1 ; O=C(Oc1coc(CN2CCSCC2)cc1=O)c1ccc([N+](=O)[O-])cc1]

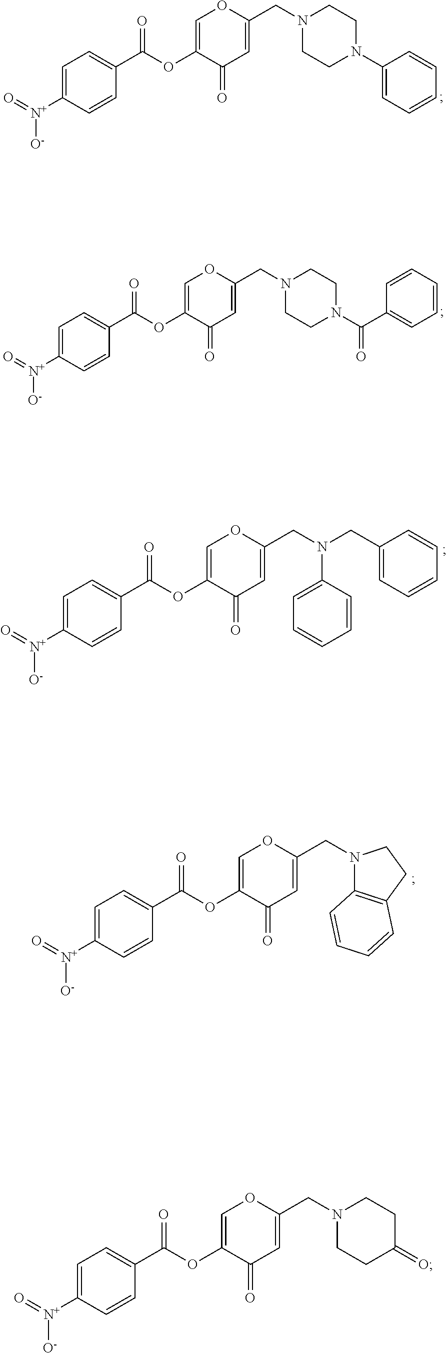 [SMILES: O=C(Oc1coc(CN2CCN(c3ccccc3)CC2)cc1=O)c1ccc([N+](=O)[O-])cc1 ; O=C(Oc1coc(CN2CCN(C(=O)c3ccccc3)CC2)cc1=O)c1ccc([N+](=O)[O-])cc1 ; O=C(Oc1coc(CN(Cc2ccccc2)c2ccccc2)cc1=O)c1ccc([N+](=O)[O-])cc1 ; O=C(Oc1coc(CN2CCc3ccccc32)cc1=O)c1ccc([N+](=O)[O-])cc1 ; O=C1CCN(Cc2cc(=O)c(OC(=O)c3ccc([N+](=O)[O-])cc3)co2)CC1]

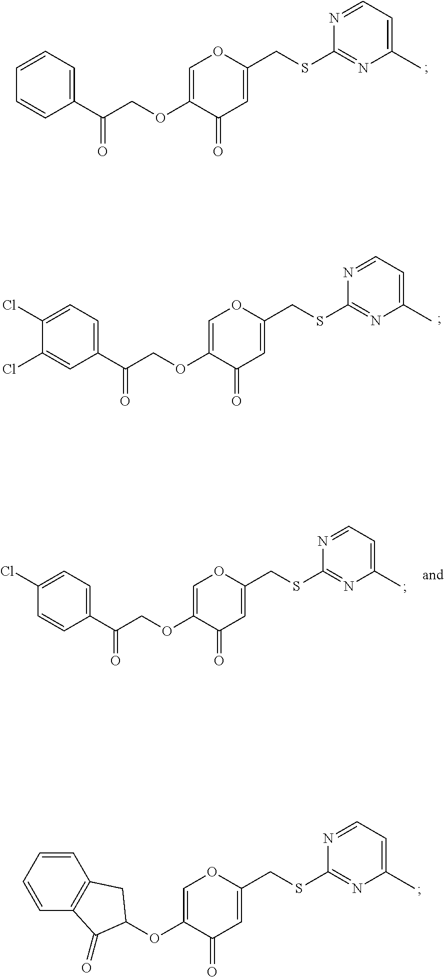 [SMILES: Cc1ccnc(SCc2cc(=O)c(OCC(=O)c3ccccc3)co2)n1 ; Cc1ccnc(SCc2cc(=O)c(OCC(=O)c3ccc(Cl)c(Cl)c3)co2)n1 ; Cc1ccnc(SCc2cc(=O)c(OCC(=O)c3ccc(Cl)cc3)co2)n1 ; Cc1ccnc(SCc2cc(=O)c(OC3Cc4ccccc4C3=O)co2)n1]

or a pharmaceutically acceptable salt, polymorph, solvate, tautomer, or N-oxide thereof.

### Methods

In one embodiment, provided is a method of inhibiting the activity of APJ or apelin signaling in a cell, inhibiting the binding of apelin to APJ, or inhibiting the activation of APJ by apelin, the method comprising contacting the cell with an effective amount of a compound as described herein. Such contacting can be in vitro or in vivo.

In one embodiment, provided is a method of treating a disease mediated by apelin in a patient in need thereof, comprising administering to the patient a therapeutically effective amount of a compound as described herein.

In one embodiment the compound is of formula (I):

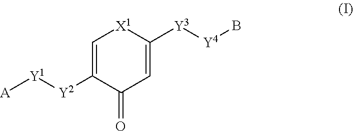

wherein:
X¹ is selected from the group consisting of —O— and —N(R⁵)—;
A is selected from the group consisting of phenyl and 5- or 6-membered heteroaryl, wherein each phenyl and heteroaryl is independently optionally substituted with from one to three R²⁰;
B is selected from the group consisting of phenyl and 5- or 6-membered heteroaryl, wherein each phenyl and heteroaryl is independently optionally substituted with from one to three R²⁰;
Y¹ and Y² are each independently selected from the group consisting of a bond, —C(O)O—, —C(O)—, —CH₂O—, —N(R¹)—, —C(R²)(R³)— and —S(O)₂—;
Y³ and Y⁴ are each independently selected from the group consisting of a bond, —S—, —O—, —N(R¹)—, —C(R²)(R³)—, —C(O)— and —S(O)₂—;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof.

In certain embodiments, the disease is selected from the group consisting of neoplasia, cardiovascular disease, peripheral vascular disease, hypertension, preeclampsia syndrome, abnormal angiogenesis, diabetes, ocular degeneration, idiopathic pulmonary fibrosis, would healing, chronic obstructive pulmonary disease, inflammatory disease such as arthritis, and inflammatory bowel disease, cardiovascular disease, avascular or ischemic insult, myocardial infarction, stroke, vaculitis, systemic or vascular sclerosis, gangrene, congelation, alopecia, eczema, ulcers, lymphedema, vascular hyperplasia, hemangioma, diabetic induce retinopathy, macular degenerative disease, psoriasis, or endometriosis.

In one embodiment, the disease is ocular degeneration.

In another embodiment, provided is a method of treating a tumor or disease caused by abnormal angiogenesis in a patient in need thereof, comprising administrating to the patient a therapeutically effective amount of a compound as provided herein (e.g., a compound of Formula (I), (II), or (III).

In one embodiment, administration of the compound modifies tumor cell growth or endothelial cell growth in the patient, thereby treating the tumor or disease caused by abnormal angiogenesis.

In certain embodiments, the tumor or disease caused by abnormal angiogenesis is cancer. However, abnormal angiogenesis is not limited to cancer. Other diseases, including macular degeneration, are linked to abnormal development of blood vessels.

### Pharmaceutical Formulations

The disclosure also provides articles of manufacture comprising packaging material and a pharmaceutical composition contained within said packaging material, wherein said packaging material comprises a label which indicates that said pharmaceutical composition can be used for treatment of disorders and wherein said pharmaceutical composition comprises a compound according to the disclosure.

The disclosure also provides pharmaceutical compositions comprising at least one compound or a pharmaceutically acceptable salt, polymorph, or solvate thereof, in an amount effective for treating a disorder, and a pharmaceutically acceptable excipient. The compositions of the disclosure may contain other therapeutic agents as described below, and may be formulated, for example, by employing one or more conventional solid or liquid excipients, as well as pharmaceutical additives of a type appropriate to the mode of desired administration (for example, binders, preservatives, stabilizers, flavors, etc.) according to techniques such as those well known in the art of pharmaceutical formulation. Additional excipients which are contemplated for use in the practice of the disclosure are those available to those of ordinary skill in the art, for example, those found in the United States Pharmacopeia Vol. XXII and National Formulary Vol. XVII, U.S. Pharmacopeia Convention, Inc., Rockville, Md. (1989), the relevant contents of which is incorporated herein by reference.

The disclosed pharmaceutical compositions may be administered by any suitable means, for example, orally, such as in the form of tablets, capsules, granules or powders; sublingually; buccally; parenterally, such as by subcutaneous, intravenous, intramuscular, intrathecal, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

In addition to primates, such as humans, a variety of other mammals can be treated according to the method of the disclosure. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The pharmaceutical compositions for the administration of the compounds of this embodiment either alone or in combination with other agents, e.g., chemotherapeutic, may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Also useful as a solubilizer is polyethylene glycol, for example. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a parenterally-acceptable diluent or solvent or cosolvent or complexing agent or dispersing agent or excipient or combination thereof, for example 1,3-butane diol, polyethylene glycols, polypropylene glycols, ethanol or other alcohols, povidones, Tweens, sodium dodecyle sulfate, sodium deoxycholate, dimethylacetamide, polysorbates, poloxamers, cyclodextrins, e.g., sulfobutyl ether f3-cyclodextrin, lipids, and excipients such as inorganic salts (e.g., sodium chloride), buffering agents (e.g., sodium citrate, sodium phosphate), and sugars (e.g., saccharose and dextrose). Among the acceptable vehicles and solvents that may be employed are water, dextrose solutions, Ringer's solutions and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration. For injection, the pharmaceutical compositions of the disclosure may be formulated in aqueous solutions, for example, in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

The compounds of the disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols. For topical use, creams, ointments, jellies, solutions or suspensions, etc., containing the compounds of the disclosure are employed. For purposes of this application, topical application shall include mouthwashes and gargles.

In the methods described herein, an appropriate dosage level will generally be about 0.01 to 500 mg per kg patient body weight per day which can be administered in single or multiple doses. The dosage level can be about 0.01 to about 250 mg/kg per day, such as 0.01 to about 100 mg/kg per day, for example, 0.01 to about 10 mg/kg per day, such as 0.04 to about 5 mg/kg per day, or about 0.5 to about 100 mg/kg per day. A suitable dosage level may be also about 0.05 to 100 mg/kg per day, or about 0.1 to 50 mg/kg per day or 1.0 mg/kg per day. Within this range the dosage may be 0.05 to 0.5, 0.5 to 5 or 5 to 50 mg/kg per day for example. The Examples section shows that one of the exemplary compounds was dosed at 0.1 mg/kg/day while another was effective at about 1.0 mg/kg/day. For oral administration, the compositions may be provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, or once or twice per day. There may be a period of no administration followed by another regimen of administration. Administration of the compounds may be closely associated with the schedule of a second agent of administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### General Synthesis

The general method for preparation of the compounds described herein is shown in the scheme below (Scheme 1). Starting from kojic acid (A), chlorination using neat thionyl chloride gave B. This was then used to alkylate a thiol to give C. Reaction with an acid chloride then provided the desired APJ antagonists D. In general yields were reasonable for this sequence and gram scale quantities of materials could be produced.

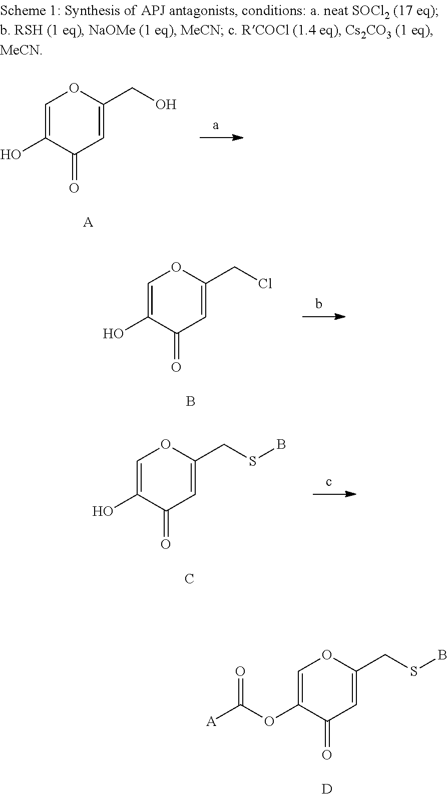

### EXAMPLES

The present methods, thus generally described, will be understood more readily by reference to the following examples, which are provided by way of illustration and are not intended to be limiting of the present methods and kits.

### Example 1

### SYNTHESIS OF 4-OXO-6-((PYRIMIDIN-2-YLTHIO)METHYL)-4H-PYRAN-3-YL-4-NITROBENZOATE (COMPOUND 6)

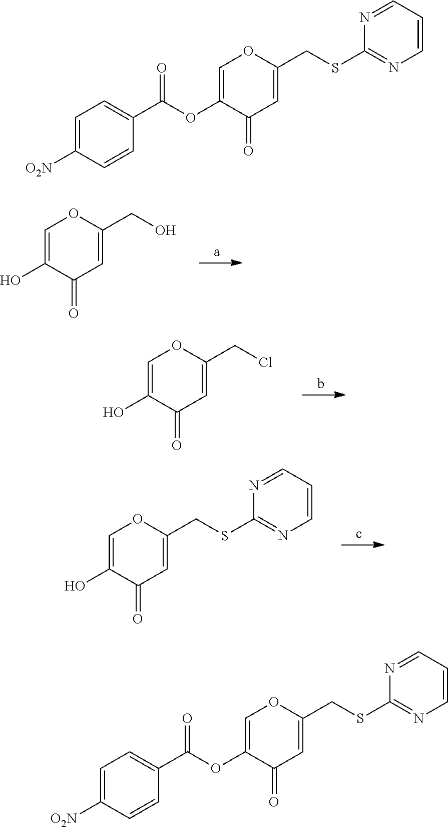 [SMILES: O=C(Oc1coc(CSc2ncccn2)cc1=O)c1ccc([N+](=O)[O-])cc1 ; O=c1cc(CO)occ1O ; O=c1cc(CCl)occ1O ; O=c1cc(CSc2ncccn2)occ1O ; O=C(Oc1coc(CSc2ncccn2)cc1=O)c1ccc([N+](=O)[O-])cc1]

Step a: A mixture of 5-hydroxy-2-(hydroxymethyl)-4H-pyran-4-one (Kojic acid) (0.55 g, 3.87 mmol) was dissolved in thionyl chloride (5 ml, 68.5 mmol) and was stirred at ambient temperature for 3 hours. Excess reagent was removed in vacuo to provide 0.61 g. (98%) of 2-(chloromethyl)-5-hydroxy-4H-pyran-4-one as an off-white solid. ¹H NMR. (500 MHz, DMSO-d6): δ (ppm) 8.13 (s, 1H), 6.57 (s, 1H), 4.66 (s, 2H).

Step b: A mixture of pyrimidine-2-thiol (161 mg, 1.433 mmol) in 2 ml methanol was treated with sodium methoxide solution (310 mg, 1.433 mmol) and stirred until dissolved. Acetonitrile (10 ml) was added followed by 2-(chloromethyl)-5-hydroxy-4H-pyran-4-one (230 mg, 1.433 mmol) and the mixture was stirred at ambient temperature for 3 hours at which time analysis by LC/MS indicated the reaction to be complete. The solvent was removed in vacuo to provide 406 mg (96%) of a yellow solid containing crude 5-hydroxy-2-((pyrimidin-2-ylthio)methyl)-4H-pyran-4-one and an equimolar amount of sodium chloride which was used without further purification. ¹H NMR. (500 MHz, CDCl₃): δ (ppm) 8.52 (d, 2H, J=4.9 Hz), 7.80 (s, 1H), 7.02 (t, 1H, J=4.8 Hz), 6.63 (s, 1H), 4.23 (s, 2H).

Step c: A mixture of 5-hydroxy-2-((pyrimidin-2-ylthio)methyl)-4H-pyran-4-one (200 mg, 0.847 mmol), cesium carbonate (276 mg, 0.847 mmol), and 4-nitrobenzoyl chloride (220 mg, 1.185 mmol) in acetonitrile (8 ml) was stirred at ambient temperature overnight. The solvent was removed in vacuo to provide a pale yellow solid, which was partitioned with approximately 20 ml of 1:1 ethyl acetate and water. The desired product remained insoluble in the biphase and was collected by filtration. The solid was dried in vacuo to yield 202 mg (62%) as a tan solid. ¹H NMR. (500 MHz, DMSO-d6): δ (ppm) 8.69 (d, 2H, J=4.8 Hz), 8.68 (s, 1H), 8.40 (d, 2H, J=8.8 Hz), 8.29 (d, 2H, J=8.8 Hz), 7.29 (t, 1H, J=4.9 Hz), 6.65 (s, 1H), 4.45 (s, 2H). ¹³C NMR. (125 MHz, DMSO-d6): δ (ppm) 171.2, 168.8, 165.9, 161.7, 158.1, 150.8, 149.9, 140.3, 133.0, 131.4, 124.2, 118.0, 114.6, 31.2.

Each of the following compounds was synthesized according to the procedure described above for Compound 6 using the appropriate starting material.

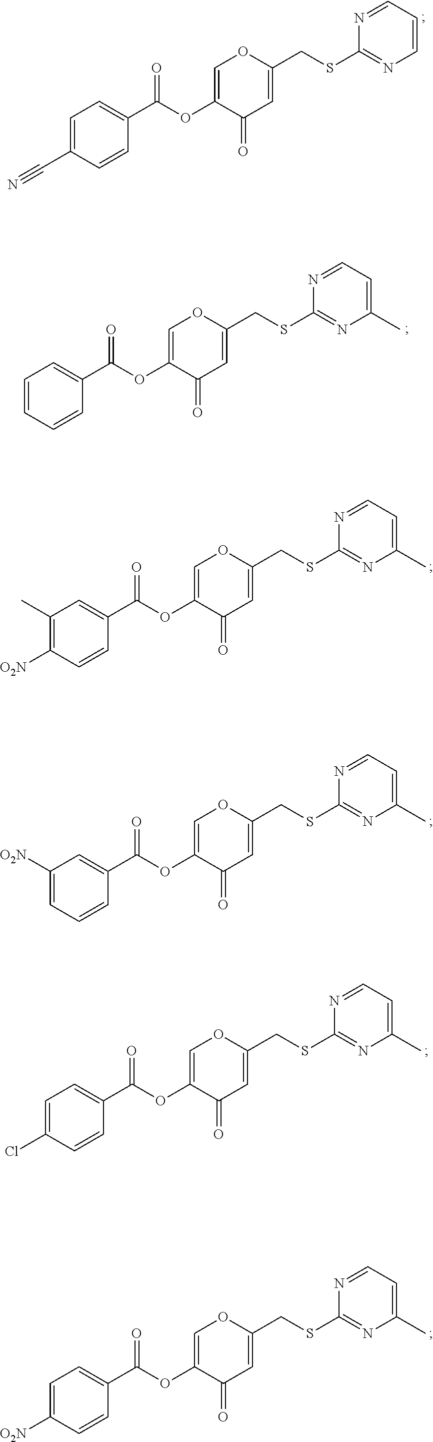

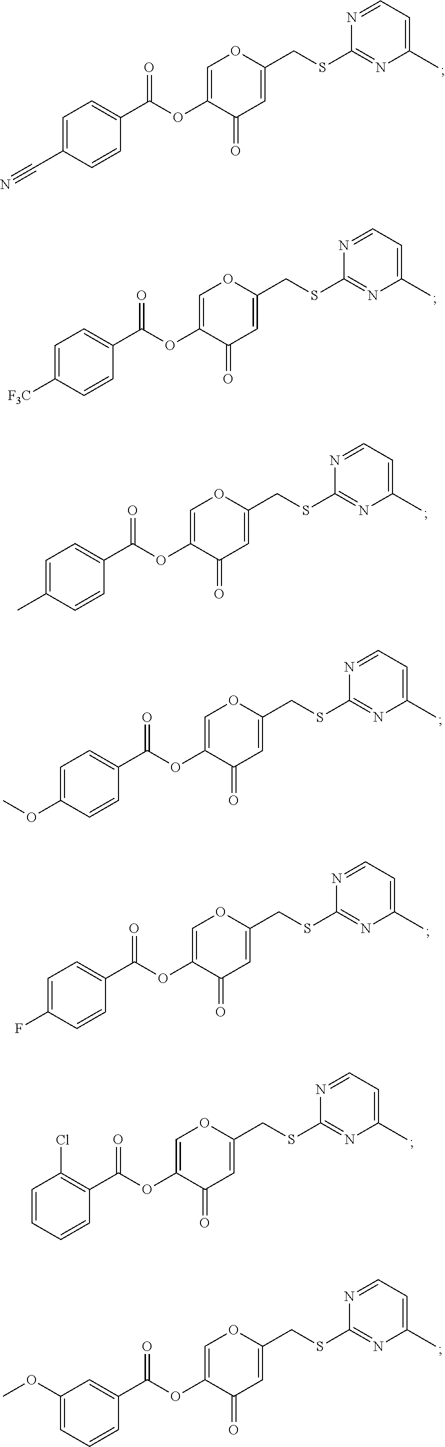

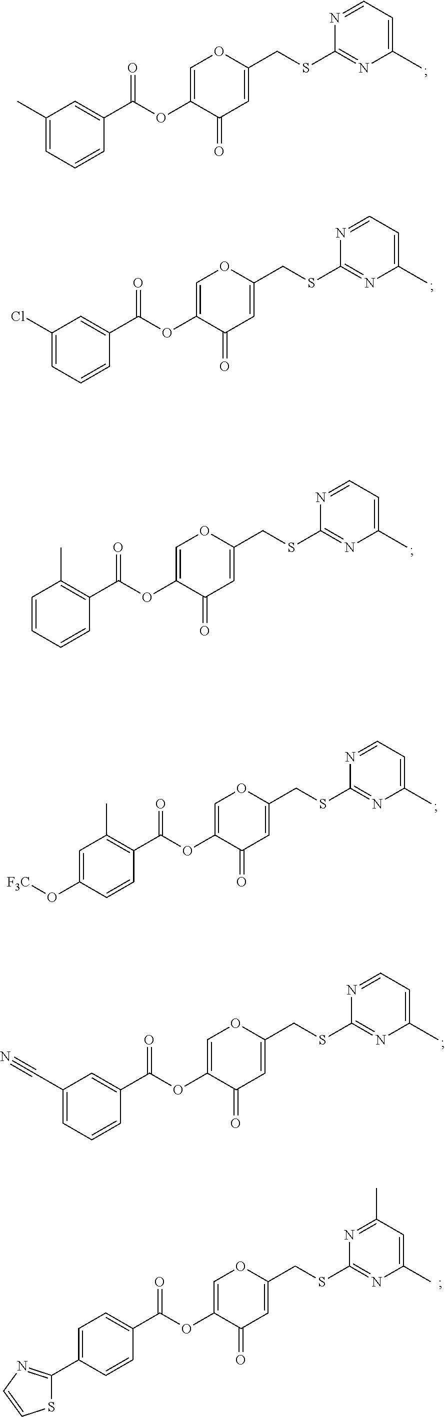

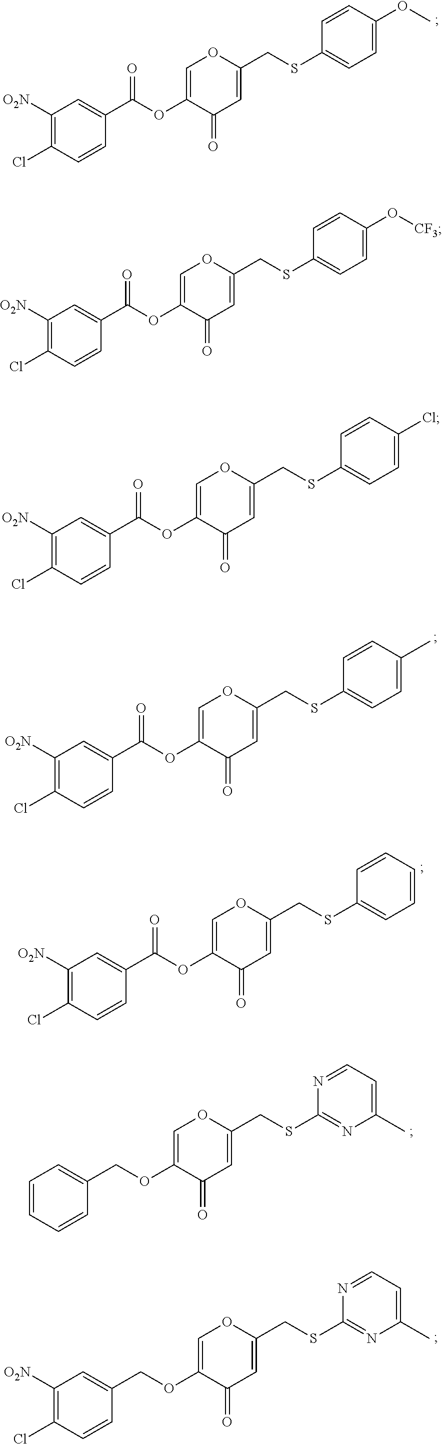

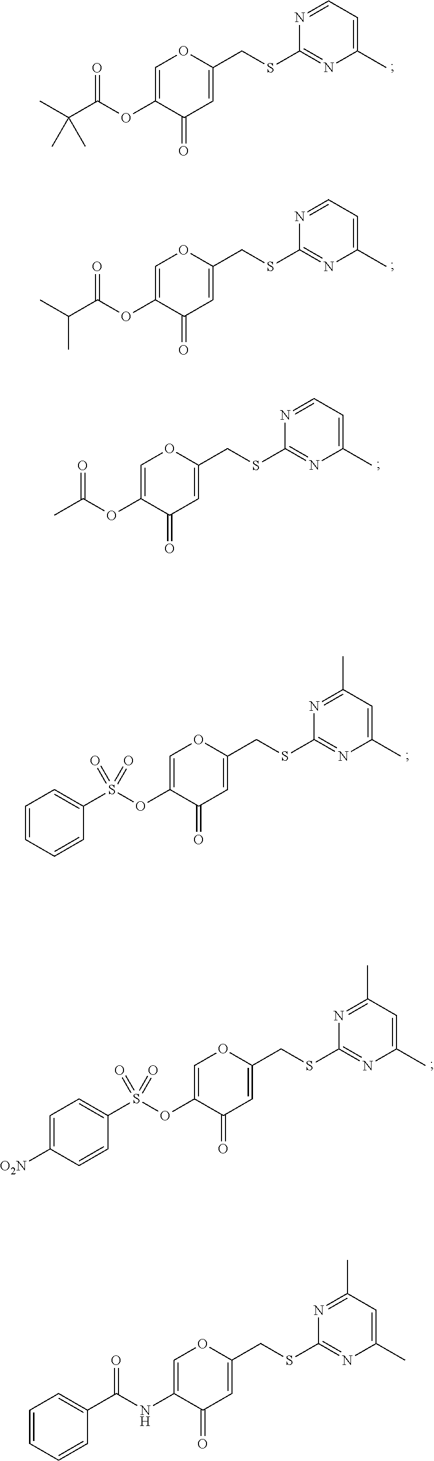

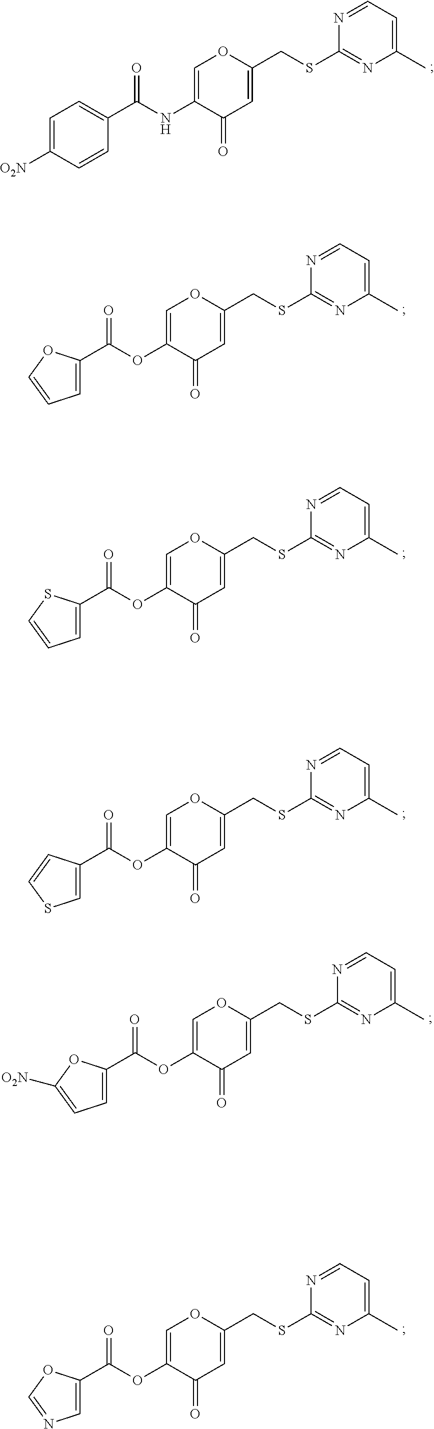 [SMILES: Cc1ccnc(SCc2cc(=O)c(NC(=O)c3ccc([N+](=O)[O-])cc3)co2)n1 ; Cc1ccnc(SCc2cc(=O)c(OC(=O)c3ccco3)co2)n1 ; Cc1ccnc(SCc2cc(=O)c(OC(=O)c3cccs3)co2)n1 ; Cc1ccnc(SCc2cc(=O)c(OC(=O)c3ccsc3)co2)n1 ; Cc1ccnc(SCc2cc(=O)c(OC(=O)c3ccc([N+](=O)[O-])o3)co2)n1 ; Cc1ccnc(SCc2cc(=O)c(OC(=O)c3cnco3)co2)n1]

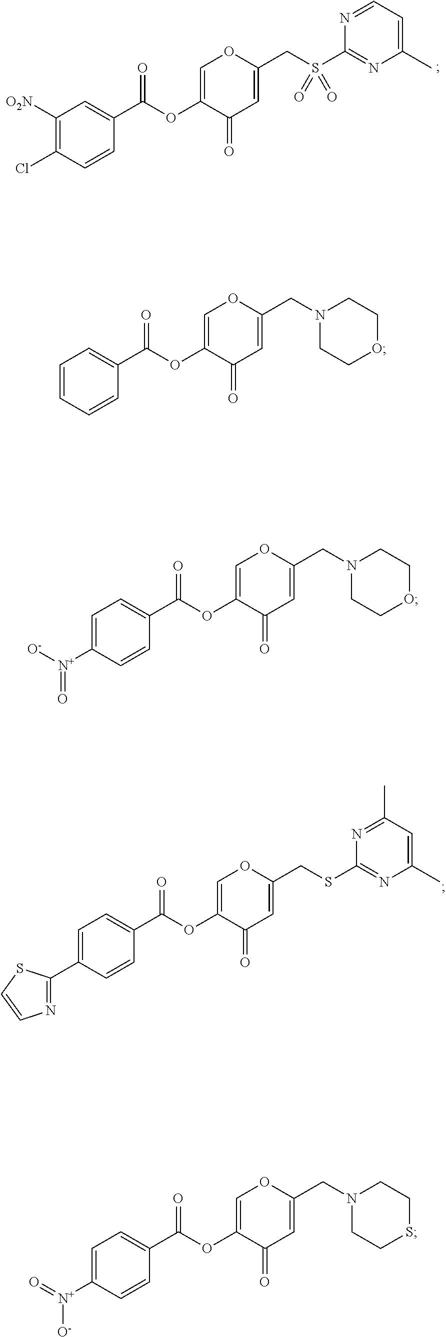 [SMILES: Cc1ccnc(S(=O)(=O)Cc2cc(=O)c(OC(=O)c3ccc(Cl)c([N+](=O)[O-])c3)co2)n1 ; O=C(Oc1coc(CN2CCOCC2)cc1=O)c1ccccc1 ; O=C(Oc1coc(CN2CCOCC2)cc1=O)c1ccc([N+](=O)[O-])cc1 ; Cc1cc(C)nc(SCc2cc(=O)c(OC(=O)c3ccc(-c4nccs4)cc3)co2)n1 ; O=C(Oc1coc(CN2CCSCC2)cc1=O)c1ccc([N+](=O)[O-])cc1]

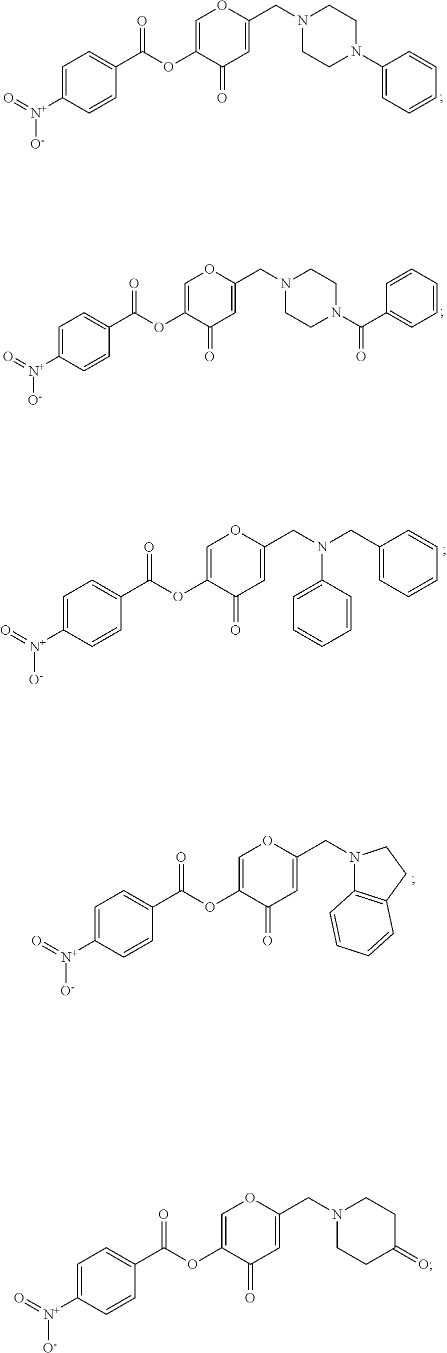 [SMILES: O=C(Oc1coc(CN2CCN(c3ccccc3)CC2)cc1=O)c1ccc([N+](=O)[O-])cc1 ; O=C(Oc1coc(CN2CCN(C(=O)c3ccccc3)CC2)cc1=O)c1ccc([N+](=O)[O-])cc1 ; O=C(Oc1coc(CN(Cc2ccccc2)c2ccccc2)cc1=O)c1ccc([N+](=O)[O-])cc1 ; O=C(Oc1coc(CN2CCc3ccccc32)cc1=O)c1ccc([N+](=O)[O-])cc1 ; O=C1CCN(Cc2cc(=O)c(OC(=O)c3ccc([N+](=O)[O-])cc3)co2)CC1]

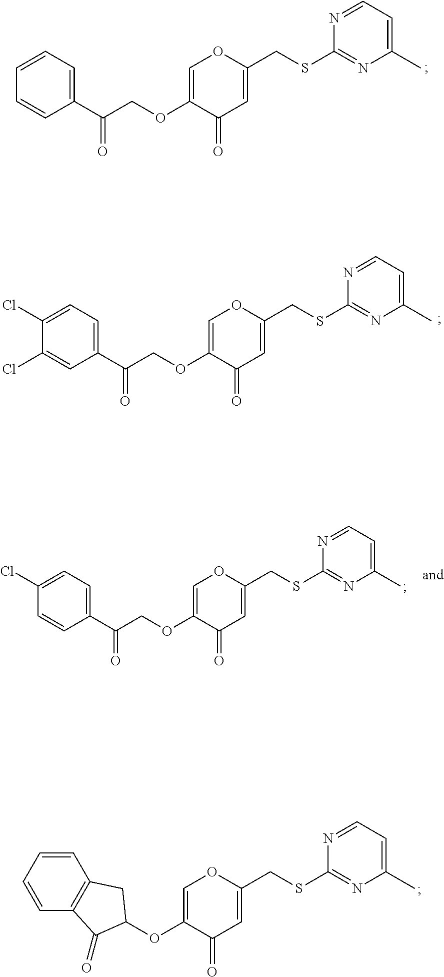 [SMILES: Cc1ccnc(SCc2cc(=O)c(OCC(=O)c3ccccc3)co2)n1 ; Cc1ccnc(SCc2cc(=O)c(OCC(=O)c3ccc(Cl)c(Cl)c3)co2)n1 ; Cc1ccnc(SCc2cc(=O)c(OCC(=O)c3ccc(Cl)cc3)co2)n1 ; Cc1ccnc(SCc2cc(=O)c(OC3Cc4ccccc4C3=O)co2)n1]

### Example 2

### BIOLOGICAL EXAMPLES

An improved potency for APJ (also known as the angiotensin II receptor-like 1 target) with 30× selectivity against the related counter target angiotensin receptor 1 (AT1) is the primary driver for compound selection and optimization. An initial full-dose response counterscreen of the scaffold selected for the SAR compounds was used to ensure that these compounds were not non-specifically inhibiting β-galactosidase activity, as the DiscoveRx primary screen is based upon the formation of a functional β-galactosidase enzyme upon β-arrestin migration subsequent to GPCR signaling.

Antagonism of apelin-13-mediated Activation of APJ by Compound 6

Cells (Angiotensin II receptor-like 1 (AGTRL-1) Cell Line (DiscoveRx, Cat#93-0250C2)) were seeded at 1000 cell/well (1536 plate, Corning) in 4 μL and grown overnight (16-18 hrs) at 37 C, 5% CO₂, 100% humidity, then 60 nL of either DMSO control or 2 mM stock test compounds in DMSO were transferred to each well, followed by 2 μL of 30 nM Apelin-13 to negative control and test compound wells, and 2 μL of assay media (F12 nutrient mix HAMs supplemented with 10% hi-FBS, 1× Penicillin/Streptomycin) to positive control wells. This yielded a final concentration of test compound of 20 μM and 1% final DMSO. The assay was incubated for 90 mins at room temperature, and then developed with 3 μL of detection reagent (PathHunter Detection Reagents (DiscoveRx, Cat#93-0001)) for 60 mins and luminescence read on a Perkin Elmer ViewLux.

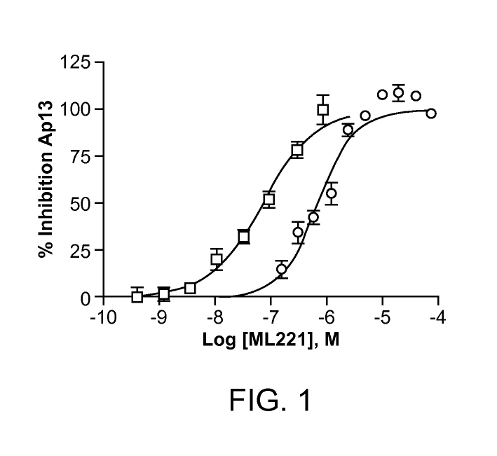

Antagonism of apelin-13-mediated activation of APJ was assessed using two complimentary assays of APJ function; inhibition of cAMP and recruitment of β-arrestin. Increasing concentrations of Compound 6 antagonized a fixed concentration of Ap13 (EC₈₀=10 nM) in both assays, with a calculated IC₅₀ equal to 0.70 μM in the cAMP assay, and 1.75 μM in the β-arrestin assay (FIG. 1).

The drug-like and ADMET properties of Compound 6 were evaluated in a detailed in vitro pharmacology panel (Table 1).

TABLE 1
ADMET properties for ML221

| Aqueous Solubility pH 7.4 | 25.5 | μg/mL |
|---|---|---|
| Plasma Protein Binding (mouse) | 99.1% | |
| Permeability (PAMPA) pH 7.4 | 271.0 × 10⁻⁶ cm/s | |
| Hepatic Microsome Stability | 4.9% | |
| Plasma Stability | 42.2% | |
| Cytotoxicity | >50 | μM |

Compound 6 is poorly soluble in aqueous media at pH 5.0/6.2/7.4, although the solubility appears pH dependent as it is almost three-fold higher in pH 7.4 than in either pH 6.2 or pH 5.0. We note that the aqueous solubility obtained at physiological pH is 5-14 fold higher than the obtained potency of the probe. In a PAMPA permeability assay, Compound 6 exhibits moderate permeability that increased with pH.

However, Compound 6 displays poor plasma and microsomal stability; it was undetectable in the plasma protein binding assay indicating that the compound is likely rapidly metabolized in plasma.

Compound 6 is rapidly metabolized in both human and mouse liver homogenates (4.2% & 4.9% remaining at 60 min). Neither the plasma nor the microsomal stability assay results are surprising given the ester linkage in this probe. Ultimately this limits the utility of this probe to in vitro studies or apelin receptor or in vivo studies using acute intravenous doses to avoid metabolism. Lastly, Compound 6 shows no toxicity (>50 μM) toward human hepatocytes.

TABLE 2
Summary of Assays*

| | Assay | Assay | Assay Detection |
|---|---|---|---|
| PubChemBioAssay Name | Type | Format | & well format |
| Summary assay for small | Summary | N/A | N/A |
| molecule antagonists of the APJ | | | |
| receptor [Summary] | | | |
| uHTS identification of small | Primary | Cell- | Luminescence- |
| molecule antagonists of the | | based | DiscoveRx β- |
| APJ receptor via a luminescent | | | arrestin & 1536 |
| beta-arrestin assay [Primary | | | |
| Screening] | | | |
| Single concentration confirma- | Confir- | Cell- | Luminescence- |
| tion of uHTS hits from a small | matory | based | DiscoveRx β- |
| molecule antagonists of the APJ | | | arrestin & 1536 |
| receptor via a luminescent | | | |
| beta-arrestin assay [Primary | | | |
| Screening] | | | |
| Dose Response confirmation | Confir- | Cell- | Luminescence- |
| of uHTS hits from a small | matory | based | DiscoveRx β- |
| molecule antagonists of the APJ | | | arrestin & 1536 |
| receptor via a luminescent | | | |
| beta-arrestin assay | | | |
| [Confirmatory] | | | |
| SAR analysis of small | Selec- | Cell- | Luminescence- |
| molecule antagonists of the APJ | tivity | based | DiscoveRx β- |
| receptor via a luminescent | SAR | | arrestin & 1536 |
| beta- arrestin assay | | | |
| [Confirmatory] | | | |
| HTS Dose response | Counter- | Cell- | Luminescence |
| counterscreen for assays | screen | based | & 1536 |
| utilizing the enzyme, beta- | | | |
| galactosidase | | | |
| [Confirmatory] | | | |
| SAR analysis of antagonists | Selec- | Cell- | Luminescence- |
| of Angiotensin II | tivity | based | DiscoveRx β- |
| Receptor Type 1 to assess | SAR | | arrestin & 1536 |
| selectivity of uHTS small | | | |
| molecule antagonists hits | | | |
| of the APJ receptor | | | |
| [Confirmatory] | | | |

*Probe type = Antagonist

All assays in Table 2, with the exception of the 3-galactosidase counterscreen, utilize the DiscoveRx PathHunter® β-arrestin assay technology. Unlike imaging or other second messenger assays, the DiscoveRx β-arrestin assay allows for a direct measure of GPCR activation by detection of β-arrestin binding to the APJ, and in the case of the counterscreen, the AT1 receptor. In this system, β-arrestin is fused to an N-terminal deletion mutant of β-galactosidase (termed the enzyme acceptor of EA) and the GPCR of interest is fused to a smaller (42 amino acids), weakly complementing fragment termed ProLink™. In cells that stably express these fusion proteins, ligand stimulation results in the interaction of β-arrestin and the ProLink-tagged GPCR, forcing the complementation of the two β-galactosidase fragments and resulting in the formation of a functional enzyme that converts substrate to detectable luminescent signal.

### APJ β-Arrestin Assays

TABLE 3
Key Reagents used for the uHTS experiments

| Reagent | Vendor |
|---|---|
| Angiotensin II receptor-like 1 (AGTRL-1) Cell Line | DiscoveRx |
| (DiscoveRx, Cat# 93-0250C2) | |
| PathHunter Detection Reagents (DiscoveRx, Cat# | DiscoveRx |
| 93-0001) | |
| Apelin-13 (Sigma-Aldrich, Cat# A6469) | Sigma-Aldrich |
| Assay media: F12 nutrient mix HAMs supplemented | Invitrogen |
| with 10% hi-FBS, 1X Penicillin/Streptomycin | |
| 1536-well, white, solid-bottom, Kalypsys | Corning |
| compatible, tissue culture treated | |

Basic Protocols:

Cells were seeded at 1000 cell/well (1536 plate) in 4 μL and grown overnight (16-18 hrs) at 37 C, 5% CO₂, 100% humidity, then 60 nL of either DMSO control or 2 mM stock test compounds in DMSO were transferred to each well, followed by 2 μL of 30 nM Apelin-13 to negative control and test compound wells, and 2 μL of assay media to positive control wells. This yielded a final concentration of test compound of 20 μM and 1% final DMSO. Assay was incubated for 90 mins at room temperature, and then developed with 3 μL of detection reagent for 60 mins and luminescence read on a Perkin Elmer ViewLux. Initial actives were retested in duplicate at the 20 μM test concentration, and reconfirmed hits retested in full dose-response for compounds from resupplied MLSMR DMSO stock solutions. All solid powder re-orders, compounds ordered via analogue-by-catalogue, and SAR submitted compounds were tested in dose response in a similar manner.

### AT1 β-Arrestin Assays

Assay Materials and Protocols:

The detailed protocols, materials and reagent recipes can be obtained from the AIDs listed, above. Some of the key reagents and assay plates used are listed below in Table 4.

TABLE 4
Key Reagents used for the uHTS experiments

| Reagent | Vendor |
|---|---|
| Angiotensin receptor 1 (AT1) Cell Line (DiscoveRx, | DiscoveRx |
| Cat# 93-0312C2) | |
| PathHunter Detection Reagents (DiscoveRx, Cat# | DiscoveRx |
| 93-0001) | |
| Angiotensin II (Sigma-Aldrich, Cat# A9525) | Sigma-Aldrich |
| Assay Media: F12 nutrient mix HAMs supplemented | Invitrogen |
| with 6% hi-FBS, 1X Penicillin/Streptomycin | |
| 1536-well, white, solid-bottom, Kalypsys | Corning |
| compatible, tissue culture treated plates | |

Basic Protocol:

Cells were seeded at 500 cell/well (1536 plate) in 5 μL and grown overnight (16-18 hrs) at 37 C, 5% CO₂, 100% humidity, then 80 mL of DMSO was transferred to control wells. Next, varying volumes of stock test compounds in DMSO were transferred to each well to achieve appropriate dose response concentrations and range. DMSO was backfilled into each test compound well to a total volume of 80 nL to achieve equal concentration of DMSO in all wells. This was followed by an addition of 1 μL of 300 nM reference agonist, Angiotensin II, to the negative and test compound wells, and 1 μL of assay media to positive control wells. The assay was incubated for 90 mins at room temperature, and then developed with 3 μL of detection reagent for 60 mins and luminescence read on a Perkin Elmer ViewLux. All MLSMR DMSO stock solutions, solid powder re-orders, compounds ordered via analogue-by-catalogue, and SAR submitted compounds were tested in dose response in a similar manner.

### β-Galactosidase Counterscreen Assay

Assay Materials:

The detailed protocols, materials and reagent recipes can be obtained from the AIDs listed, above. Some of the key reagents and assay plates used are listed below in Table 5.

TABLE 5
Key Reagents used for the uHTS experiments

| Reagent | Vendor |
|---|---|
| PathHunter Detection Reagents (DiscoveRx, Cat# | DiscoveRx |
| 93-0001) | |
| β-Galactosidase (Sigma-Aldrich, Cat# A4155) | Sigma-Aldrich |
| Assay Media: F12 nutrient mix HAMs supplemented | Invitrogen |
| with 10% hi-FBS, 1X Penicillin/Streptomycin | |
| 1536-well, white, solid-bottom, Kalypsys | Corning |
| compatible, tissue culture treated plates | |

Basic Protocol:

First, 5 μL of 0.03 U/mL β-galactosidase in assay media is added to the negative control and test compound wells of assay plate. To positive control wells are added 5 μL of assay media. Then 80 nL of DMSO was transferred to control wells. Next, varying volumes of stock test compounds in DMSO were transferred to each well to achieve appropriate dose response concentrations and range. DMSO was backfilled into each test compound well to a total volume of 80 nL to achieve equal concentration of DMSO in all wells. Next, 2.5 μL of detection reagents are added to all wells of assay plate. Assay was incubated for 15 mins at room temperature and then read on a Perkin Elmer ViewLux using a luminescence protocol.

### Profiling Assays

The nominated probe was evaluated in a detailed in vitro pharmacology screen as shown in Table 6.

TABLE 6
Summary of in vitro ADME Properties
of APJ Antagonist probe Compound 6

| | Aqueous | Aqueous | |
|---|---|---|---|
| | Solubility | Solubility in 1x | PAMPA Pe |
| | (μg/mL) | PBS (μg/mL) | (×10⁻⁶ cm/s) |
| | (pH 5.0/6.2/7.4) | pH 7.4 | Donor pH: 5.0/6.2/7.4 |
| Probe | [μM]^{a} | [μM] | Acceptor pH: 7.4 |
| Compound | 3.0/3.6/9.8 | 3.6 | 195/240/271 |
| 6 | [7.8/9.3/25] | [9.3] | |
| | Plasma Stability | | |
| Plasma Protein | (% Remaining @3 hrs) | | |
| Binding | Human/Mouse | Hepatic | |
| (% Bound) | Plasma:1x PBS, | Microsome | Hepatic |
| Human/Mouse | pH 7.4, 1:1 | Stability | Toxicity |
| 1 μM | 1x PBS, pH 7.4 | Human/Mouse | LC50 (μM) |
| ND/ND | 0.26/0.88 | 4.19/4.89 | >50 |
| | 46.97/42.18 | | |

^{a}Solubility also expressed in molar units (μM) as indicated in italicized [bracketed values], in addition to more traditional μg/mL units.

Compound 6 is poorly soluble in aqueous media at pH 5.0/6.2/7.4, although the solubility appears pH dependent as it is almost three-fold higher in pH 7.4 than in either pH6.2 or pH5.0. We note that the aqueous solubilities obtained at physiological pH are 5-14 fold higher than the obtained potency of the probe.

The PAMPA (Parallel Artificial Membrane Permeability Assay) assay is used as an in vitro model of passive, transcellular permeability. An artificial membrane immobilized on a filter is placed between a donor and acceptor compartment. At the start of the test, drug is introduced in the donor compartment. Following the permeation period, the concentration of drug in the donor and acceptor compartments is measured using UV spectroscopy. Consistent with its solubility data, Compound 6 exhibits moderate permeability that increased with pH.

Plasma protein binding is a measure of a drug's efficiency to bind to the proteins within blood plasma. The less bound a drug is, the more efficiently it can traverse cell membranes or diffuse. Highly plasma protein bound drugs are confined to the vascular space, thereby having a relatively low volume of distribution. In contrast, drugs that remain largely unbound in plasma are generally available for distribution to other organs and tissues. Compound 6 was undetectable in the plasma protein binding assay indicating that the compound is likely rapidly metabolized in plasma.

Plasma stability is a measure of the stability of small molecules and peptides in plasma and is an important parameter, which strongly can influence the in vivo efficacy of a test compound. Drug candidates are exposed in plasma to enzymatic processes (proteinases, esterases), and they can undergo intramolecular rearrangement or bind irreversibly (covalently) to proteins. Compound 6 shows very poor stability in human plasma (<1% remaining) after 3 hr. This data explains the lack of compound detected in the plasma protein binding assay, as that assay involves an 18 h incubation.

The microsomal stability assay is commonly used to rank compounds according to their metabolic stability. This assay addresses the pharmacologic question of how long the parent compound will remain circulating in plasma within the body. Compound 6 shows poor stability (4.2% & 4.9% remaining at 60 min) in both human and mouse liver homogenates. Neither the plasma nor the microsomal stability assay results are surprising given the ester linkage in this probe. Attempts to replace the ester with a more stable functional group resulted in inactive compounds, although replacements were not widely investigated. Ultimately this limits the utility of this probe to in vitro studies or apelin receptor or in vivo studies using acute intravenous doses to avoid metabolism. Compound 6 shows no toxicity (>50 μM) toward human hepatocytes.

Profiling against other GPCRs. The probe, Compound 6, was submitted to the Psychoactive Drug Screening Program (PDSP) at the University of North Carolina (PDSP, Bryan Roth, PI) and the data against a GPCR binding assay panel is shown in FIG. 3. Overall the compound shows a relatively clean binding profile, with the only significant activity at the kappa opioid receptor.

### Additional Probe Characterization Assays.

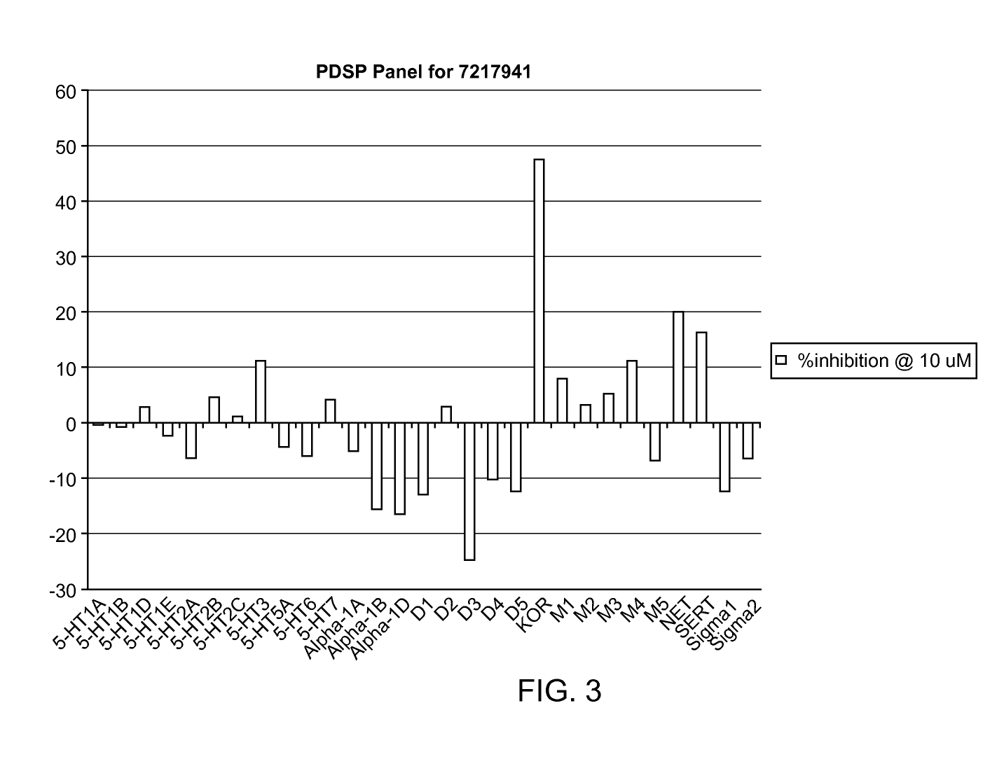

In addition to the primary assay of APJ β-arrestin recruitment, the ability of Compound 6 to antagonize APJ was further tested in a cellular assay of intracellular cAMP. Briefly, CHO-K1 cells overexpressing APJ were stimulated with forskolin (15 μM) to increase intracellular cAMP, and subsequently exposed to a range of concentrations of apelin-13, in the presence or absence of Compound 6. Intracellular cAMP, was measured using the HitHunter XS cAMP assay kit (DiscoveRx. Freemont Calif.), and the results of this study are shown in FIG. 4. Compound 6 blocked the effects of apelin-13 on intracellular cAMP (IC₅₀=38 nM). Further, it was observed that Compound 6, may act as an inverse agonist of APJ, reducing the basal intracellular cAMP.

A representative dose response curve for Compound 6 in the cAMP assay is shown in FIG. 4. This graph depicts the mean percent activity (±SEM) of forskolin induced increased cAMP representative of an experiment that was repeated three times with each data point performed in triplicate. Curve fit analysis was performed using nonlinear regression with a 4-point logistic fit (GraphPad Prism5).

The ability of Compound 6 to antagonize APJ receptor internalization after exposure to fixed concentration of apelin-13 (6.23 nM) was determined. As expected, Compound 6 antagonized the ability of apelin-13 to induce APJ receptor internalization.

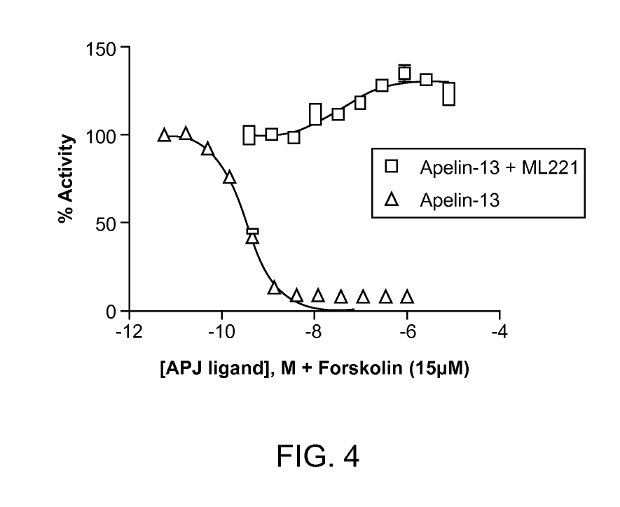

A representative dose response curve for Compound 6 in the APJ receptor internalization assay is shown in FIG. 5. This graph depicts the mean percent inhibition (±SEM) of apelin-13 mediated receptor internalization. Representative of an experiment that was repeated three times with each data point performed in triplicate. Curve fit analysis was performed using nonlinear regression with a 4-point logistic fit (GraphPad Prism5).

### Summary of Screening Results

A library of approximately 330,000 compounds was tested in the APJ DiscoveRx β-Arrestin primary screen (PubChem AID 2766; ref 32). Upon data analysis, 1064 hits with activity>50% at a single concentration point of 10 μM were identified. Liquid samples were then ordered through the MLSMR and 948 compounds were received.

The compound solutions resupplied by the MLSMR were first confirmed in 10 μM single-point duplicate in the APJ DiscoveRx β-Arrestin primary assay. Of these, 622 compounds were confirmed to have at least 50% activity at a 10 μM assay concentration. These were further triaged for direct 3-galactosidase inhibition and an additional 237 compounds were eliminated.

The remaining 385 confirmed compounds were next tested in dose response in the primary APJ DiscoveRx β-arrestin primary assay to obtain IC₅₀ values and these were rank ordered for potency: 67 compounds met probe criteria (IC₅₀=1-5 μM), 62 additional compounds had IC₅₀ between 5-10 μM, and 86 compounds were less potent (IC₅₀ between 10-20 μM). To eliminate compounds acting through non-specific inhibition of the assay reporter, the activity of the best scaffolds was assessed in a β-galactosidase counterscreen assay. The best scaffolds were also subjected to a counterscreen of the closely related angiotensin II type 1 (AT1) receptor to determine selectivity for APJ. Ultimately, only a single scaffold that was potent and selective against AT1 was identified.

TABLE 7
SAR Analysis of APJ Antagonists

| 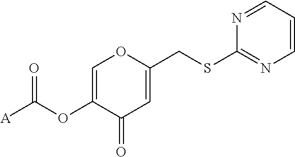 | | Potency IC₅₀ (μM) Ave. ± S.E.M. (stdv/sqrt (n)) (n = 4) if number of replicates is different than the default it is noted in parentheses. SI = Selectivity Index: (IC₅₀ AT1)/(IC₅₀ APJ) | | |
|---|---|---|---|---|
| Compound No. | A | APJ | AT1 | SI |
| 1 | phenyl | >79 | >79 | ND |
| 2 | 3-NO₂-4-Cl-phenyl | 6.20 ± 0.77 | >79 | >13 |
| 3 | 3-NO₂-4-Me-phenyl | 7.25 ± 0.75 | >79 | >11 |
| | | 10.90 ± 1.41 | >79 | >7.2 |
| 4 | 3-NO₂-phenyl | 75.70 ± 3.30 (2) | >79 | ND |
| 5 | 4-Cl-phenyl | 54.50 ± 3.12 (2) | >79 | ND |
| 6 | 4-NO₂-phenyl | 2.16 ± 0.96 | >79 | >37 |
| | | 1.75 ± 0.19 | >79 | >45 |
| 7 | 4-CN-phenyl | 6.59 ± 2.87 | >79 | >12 |
| | | 9.77 ± 0.60 | >79 | >8 |
| 8 | 4-CF₃-phenyl | 29.00 ± 4.94 (3) | >79 | ND |
| 9 | 4-Br-phenyl | 18.20 ± 3.36 (7) | >79 | ND |
| 10 | 4-Me-phenyl | >79 | >79 | ND |
| 11 | 4-OMe-phenyl | >79 | >79 | ND |
| 12 | 4-OEt-phenyl | >79 | >79 | ND |
| 13 | 4-OiPr-phenyl | >79 | >79 | ND |
| 14 | 4-OBu-phenyl | >79 | >79 | ND |
| 15 | 4-tBu-phenyl | >79 | >79 | ND |
| 16 | 4-F-phenyl | >79 | >79 | ND |
| | | >79 | >79 | |
| 17 | 4-SO₂NMe₂-phenyl | >79 | >79 | ND |
| 18 | 4-SO₂N(CH₂)₄-phenyl | >79 | >79 | ND |
| 19 | 3,4-diMe-phenyl | >79 | >79 | ND |
| 20 | 2-NO₂-5-Cl-phenyl | >79 | >79 | ND |
| 21 | 2-Cl-5-NO₂-phenyl | 51.00 ± 4.07 (4) | 60.10 ± 1.35 (3) | ND |
| 22 | 2-Me-3-NO₂-phenyl | 72.2 (1) | >79 | ND |
| 23 | 4-Ph-phenyl | >79 | >79 | ND |
| 24 | 4-SO₂NEt₂-phenyl | >79 | >79 | ND |
| 25 | 4-SO₂N(CH₂)₅-phenyl | >79 | >79 | ND |
| 26 | 4-(morpholinyl-SO₂)-phenyl | >79 | >79 | ND |
| 27 | 2-NO₂-4,5-diOMe-phenyl | >79 | >79 | ND |

TABLE 8
SAR Analysis of APJ Antagonists

| 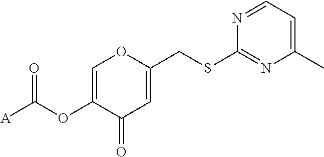 | | Potency IC₅₀ (μM) Ave. ± S.E.M. (stdv/sqrt (n)) (n = 4) if number of replicates is different than the default it is noted in parentheses. SI = Selectivity Index: (IC₅₀ AT1)/(IC₅₀ APJ) | | |
|---|---|---|---|---|
| Compound No. | A | APJ | AT1 | SI |
| 28 | phenyl | >79 | >79 | ND |
| 29 | 3-NO₂-4-Cl-phenyl | 4.77 ± 0.56 | >79 | >17 |
| | | 1.75 ± 0.14 | >79 | >45 |
| 30 | 3-NO₂-4-Me-phenyl | 4.70 ± 0.91 (3) | >79 | >17 |
| 31 | 3-NO₂-phenyl | 59.20 ± 4.77 (2) | >79 | ND |
| 32 | 4-Cl-phenyl | >79 | >79 | ND |
| 33 | 4-NO₂-phenyl | 3.11 ± 0.68 | >79 | >25 |
| | | 4.32 ± 1.29 | >79 | >23 |
| 34 | 4-CN-phenyl | 8.51 ± 1.22 | >79 | >9 |
| | | 11.50 ± 1.35 | >79 | >7 |
| 35 | 4-CF₃-phenyl | 20.6 (1) | >79 | ND |
| 36 | 4-Me-phenyl | >79 | >79 | ND |
| 37 | 4-OMe-phenyl | >79 | >79 | ND |
| 38 | 4-F-phenyl-phenyl | >79 | >79 | ND |
| 39 | 4-SO₂N(CH₂)₄-phenyl | 39.90 ± 8.65 | >79 | ND |
| 40 | 2-NO₂-5-Cl-phenyl | 58.80 ± 6.30 (2) | >79 | ND |
| 41 | 2-Cl-5-NO₂-phenyl | 74.10 (1) | >79 | ND |
| 42 | 2-Cl-phenyl | >59 | >79 | ND |
| 43 | 3-OMe-phenyl | >79 | >79 | ND |
| 44 | 3-Me-phenyl | >79 | >79 | ND |
| 45 | 3-Cl-phenyl | >79 | >79 | ND |
| 46 | 2-Me-phenyl | >79 | >79 | ND |
| 47 | 4-OCF₃-phenyl | >79 | >79 | ND |
| 48 | 3-CN-phenyl | >79 | >79 | ND |

TABLE 9
SAR Analysis of APJ Antagonists

| 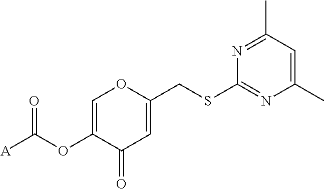 | | Potency IC₅₀ (μM) Ave. ± S.E.M. (stdv/sqrt (n)) (n = 4) if number of replicates is different than the default it is noted in parentheses. SI = Selectivity Index: (IC₅₀ AT1)/(IC₅₀ APJ) | | |
|---|---|---|---|---|
| Compound No. | A | APJ IC₅₀ | AT1 IC₅₀ | SI |
| 49 | 3-NO₂-4-Cl-phenyl | 5.45 ± 1.47 | >79 | >15 |
| 50 | 3-NO₂-4-Me-phenyl | 25.30 ± 5.80 | >79 | >3 |
| 51 | 4-SO₂NMe₂-phenyl | >79 | >79 | ND |
| 52 | 2-NO₂-5-Cl-phenyl | >79 | 49.70 ± 6.74 (6) | ND |
| 53 | 2-Cl-5-NO₂-phenyl | 44.45 ± 0.25 (2) | 49.70 ± 6.74 (6) | ND |
| 54 | 2-Me-3-NO₂-phenyl | >79 | >79 | ND |
| 55 | 4-(2-thiazolyl)-phenyl | >79 | >79 | ND |

TABLE 10
SAR Analysis of APJ Antagonists

| 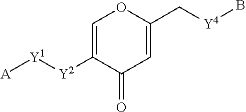 | | | Potency IC₅₀ (μM) Ave. ± S.E.M. (stdv/sqrt (n)) (n = 4) if number of replicates is different than the default it is noted in parentheses. SI = Selectivity Index: (IC₅₀ AT1)/(IC₅₀ APJ) | | |
|---|---|---|---|---|---|
| Compound No. | A—Y¹—Y²— | —Y⁴—B | APJ | AT1 | SI |
| 56 | HO— | 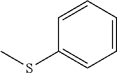 | >79 | >79 | ND |
| 57 | HO— | 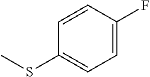 | >79 | >79 | ND |
| 58 | HO— | 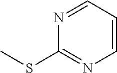 | >79 | >79 | ND |
| 59 | HO— | 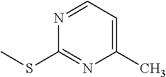 | >79 | >79 | ND |
| 60 | HO— | 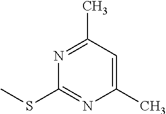 | >79 | >79 | ND |
| 61 | 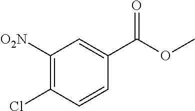 | 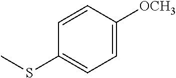 | 8.27 ± 1.75 | >79 | >9 |
| 62 | 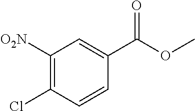 | 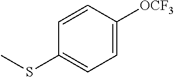 | 24.00 ± 5.45 | >79 | >3 |
| 63 | 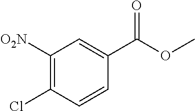 | 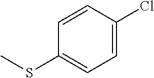 | 4.53 ± 0.66 | >79 | >17 |
| 64 | 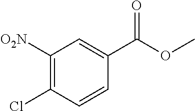 | 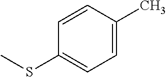 | 12.80 ± 0.35 | >79 | >6 |
| 65 | 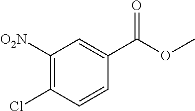 | 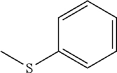 | 9.61 ± 3.02 | >79 | >8 |
| 66 | 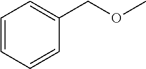 | 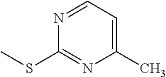 | >79 | >79 | ND |
| 67 | 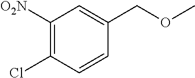 | 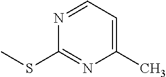 | >79 | >79 | ND |
| 68 | 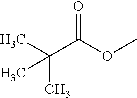 | 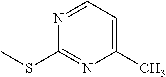 | >79 | >79 | ND |
| 69 | 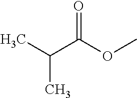 | 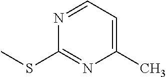 | >79 | >79 | ND |
| 70 | 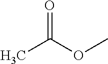 | 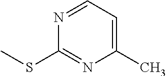 | >79 | >79 | ND |
| 71 | 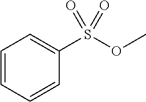 | 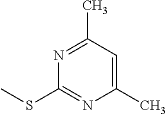 | >79 | >79 | ND |
| 72 | 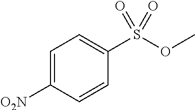 | 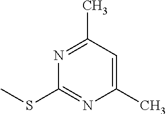 | >79 | >79 | ND |
| 73 | 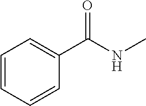 | 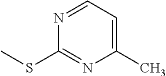 | >79 | >79 | ND |
| 74 | 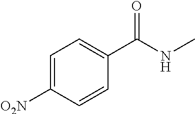 | 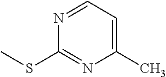 | >79 | >79 | ND |
| 75 | 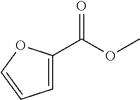 | 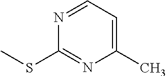 | >79 | >79 | ND |
| 76 | 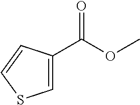 | 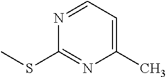 | >79 | >79 | ND |
| 77 | 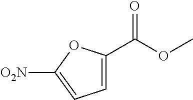 | 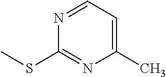 | >79 | >79 | ND |
| 78 | 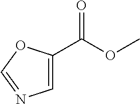 | 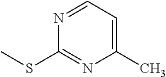 | >79 | >79 | ND |
| 79 | 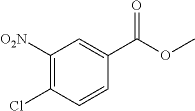 | 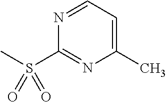 | 3.21 ± 0.46 | >79 | >25 |

### Example 3

### PROFILING AGAINST OTHER GPCRS

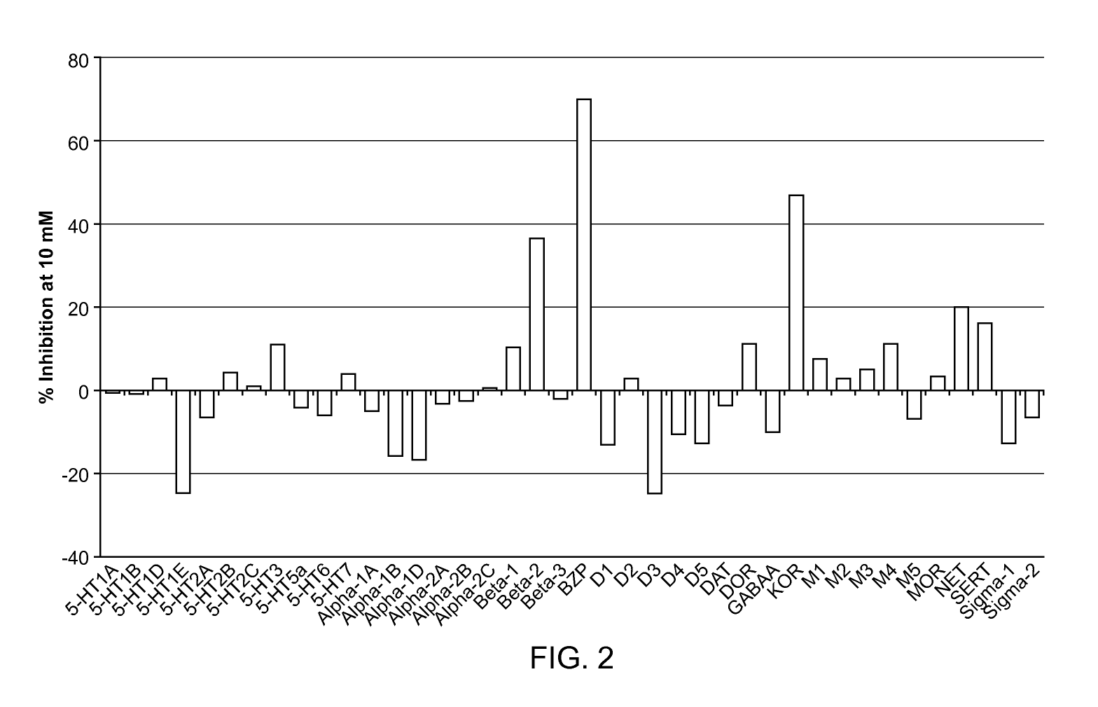

Compound 6 was submitted to the Psychoactive Drug Screening Program (PDSP) at the University of North Carolina and the data against a GPCR binding assay panel is shown in FIG. 2. Overall the compound shows a relatively clean binding profile, with the only significant activity at the kappa opioid and the benzodiazepinone receptors.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The disclosures illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the disclosures embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

Other embodiments are within the following claims. In addition, where features or aspects of the disclosure are described in terms of Markush groups, those skilled in the art will recognize that the disclosure is also thereby described in terms of any individual member or subgroup of members of the Markush group.

The following references are cited herein to aid in a more complete understanding of the present disclosure:
1. Fukusumi, S., Hinuma, S., Kitada, C., Kurokawa, T., Onda, H., and Fujino, M. (1998) Biochem Biophys Res Commun 251, 471-476
2. Han, S., Wang, G., Qi, X., Englander, E. W., and Greeley, G. H., Jr. (2008) Am J Physiol Gastrointest Liver Physiol 295, G1068-1078
3. Han, S., Wang, G., Qi, X., Lee, H. M., Englander, E. W., and Greeley, G. H., Jr. (2008) Am J Physiol Regul Integr Comp Physiol 294, R1832-1839
4. Han, S., Wang, G., Qiu, S., de la Motte, C., Wang, H. Q., Gomez, G., Englander, E. W., and Greeley, G. H., Jr. (2007) Regul Pept 142, 131-137
5. Susaki, E., Wang, G., Cao, G., Wang, H. Q., Englander, E. W., and Greeley, G. H., Jr. (2005) Regulatory peptides 129, 37-41
6. Wang, G., Anini, Y., Wei, W., Qi, X., AM, O. C., Mochizuki, T., Wang, H. Q., Hellmich, M. R., Englander, E. W., and Greeley, G. H., Jr. (2004) Endocrinology 145, 1342-1348
7. Mitra, A., Katovich, M. J., Mecca, A., and Rowland, N. E. (2006) Physiol Behav 89, 221-225
8. O'Shea, M., Hansen, M. J., Tatemoto, K., and Morris, M. J. (2003) Nutr Neurosci 6, 163-167
9. Reaux, A., De Mota, N., Skultetyova, I., Lenkei, Z., El Messari, S., Gallatz, K., Corvol, P., Palkovits, M., and Llorens-Cortes, C. (2001) J Neurochem 77, 1085-1096
10. Roberts, E. M., Newson, M. J., Pope, G. R., Landgraf, R., Lolait, S. J., and O'Carroll, A. M. (2009) J Endocrinol 202, 453-462
11. Taheri, S., Murphy, K., Cohen, M., Sujkovic, E., Kennedy, A., Dhillo, W., Dakin, C., Sajedi, A., Ghatei, M., and Bloom, S. (2002) Biochem Biophys Res Commun 291, 1208-1212
12. Attane, C., Daviaud, D., Dray, C., Dusaulcy, R., Masseboeuf, M., Prevot, D., Carpene, C., Castan-Laurell, I., and Valet, P. (2011) Journal of molecular endocrinology 46, 21-28
13. Boucher, J., Masri, B., Daviaud, D., Gesta, S., Guigne, C., Mazzucotelli, A., Castan-Laurell, I., Tack, I., Knibiehler, B., Carpene, C., Audigier, Y., Saulnier-Blache, J. S., and Valet, P. (2005) Endocrinology 146, 1764-1771
14. Dray, C., Knauf, C., Daviaud, D., Waget, A., Boucher, J., Buleon, M., Cani, P. D., Attane, C., Guigne, C., Carpene, C., Burcelin, R., Castan-Laurell, I., and Valet, P. (2008) Cell Metab 8, 437-445
15. Duparc, T., Colom, A., Cani, P. D., Massaly, N., Rastrelli, S., Drougard, A., Le Gonidec, S., Mouledous, L., Frances, B., Leclercq, I., Llorens-Cortes, C., Pospisilik, J. A., Delzenne, N. M., Valet, P., Castan-Laurell, I., and Knauf, C. (2011) Antioxidants & redox signaling 15, 1477-1496
16. Yue, P., Jin, H., Aillaud, M., Deng, A. C., Azuma, J., Asagami, T., Kundu, R. K., Reaven, G. M., Quertermous, T., and Tsao, P. S. (2010) Am J Physiol Endocrinol Metab 298, E59-67
17. Hashimoto, T., Kihara, M., Ishida, J., Imai, N., Yoshida, S., Toya, Y., Fukamizu, A., Kitamura, H., and Umemura, S. (2006) Arterioscler Thromb Vasc Biol 26, 1267-1272
18. Jia, Y. X., Lu, Z. F., Zhang, J., Pan, C. S., Yang, J. H., Zhao, J., Yu, F., Duan, X. H., Tang, C. S., and Qi, Y. F. (2007) Peptides 28, 2023-2029
19. Katugampola, S. D., Maguire, J. J., Kuc, R. E., Wiley, K. E., and Davenport, A. P. (2002) Can J Physiol Pharmacol 80, 369-374
20. Lee, D. K., Cheng, R., Nguyen, T., Fan, T., Kariyawasam, A. P., Liu, Y., Osmond, D. H., George, S. R., and O'Dowd, B. F. (2000) J Neurochem 74, 34-41
21. Tatemoto, K., Takayama, K., Zou, M. X., Kumaki, I., Zhang, W., Kumano, K., and Fujimiya, M. (2001) Regul Pept 99, 87-92
22. Chun, H. J., Ali, Z. A., Kojima, Y., Kundu, R. K., Sheikh, A. Y., Agrawal, R., Zheng, L., Leeper, N. J., Pearl, N. E., Patterson, A. J., Anderson, J. P., Tsao, P. S., Lenardo, M. J., Ashley, E. A., and Quertermous, T. (2008) J Clin Invest 118, 3343-3354
23. Hashimoto, T., Kihara, M., Imai, N., Yoshida, S., Shimoyamada, H., Yasuzaki, H., Ishida, J., Toya, Y., Kiuchi, Y., Hirawa, N., Tamura, K., Yazawa, T., Kitamura, H., Fukamizu, A., and Umemura, S. (2007) Am J Pathol 171, 1705-1712
24. Li, W. W., Niu, W. Q., Zhang, Y., Wu, S., Gao, P. J., and Zhu, D. L. (2009) J Hypertens 27, 1194-1201
25. Niu, W., Wu, S., Zhang, Y., Li, W., Ji, K., Gao, P., and Zhu, D. (2010) J Hypertens 28, 1854-1861
26. Przewlocka-Kosmala, M., Kotwica, T., Mysiak, A., and Kosmala, W. (2011) J Hypertens
27. Chong, K. S., Gardner, R. S., Morton, J. J., Ashley, E. A., and McDonagh, T. A. (2006) Eur J Heart Fail 8, 355-360
28. Foldes, G., Horkay, F., Szokodi, I., Vuolteenaho, O., Ilves, M., Lindstedt, K. A., Mayranpaa, M., Sarman, B., Seres, L., Skoumal, R., Lako-Futo, Z., deChatel, R., Ruskoaho, H., and Toth, M. (2003) Biochem Biophys Res Commun 308, 480-485
29. Meral, C., Tascilar, E., Karademir, F., Tanju, I. A., Cekmez, F., Ipcioglu, O. M., Ercin, C. N., Gocmen, I., and Dogru, T. (2010) J Pediatr Endocrinol Metab 23, 497-502
30. Erdem, G., Dogru, T., Tasci, I., Sonmez, A., and Tapan, S. (2008) Exp Clin Endocrinol Diabetes 116, 289-292
31. Kadoglou, N. P., Tsanikidis, H., Kapelouzou, A., Vrabas, I., Vitta, I., Karayannacos, P. E., Liapis, C. D., and Sailer, N. Metabolism 59, 373-379

What is claimed is:

1. A method of inhibiting the activity of APJ in a cell, comprising contacting the cell with an effective amount of a compound of formula (I): 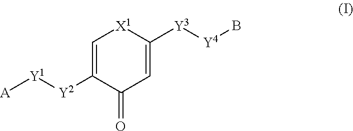
wherein:
X¹ is selected from the group consisting of —O— and —N(R⁵)—;
A is selected from the group consisting of alkyl, phenyl and 5- or 6-membered heteroaryl, wherein each phenyl and heteroaryl is independently optionally substituted with from one to three R²⁰;
B is selected from the group consisting of phenyl and 5- or 6-membered heteroaryl, wherein each phenyl and heteroaryl is independently optionally substituted with from one to three R²⁰;
Y¹ and Y² are each independently selected from the group consisting of a bond, —C(O)O—, —C(O)—, —CH₂O—, —N(R¹)—, —C(R²)(R³)— and —S(O)₂—;
Y³ and Y⁴ are each independently selected from the group consisting of a bond, —S—, —O—, —N(R¹)—, —C(R²)(R³)—, —C(O)— and —S(O)₂—;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof.

2. The method of claim 1, wherein the compound of formula (I) is represented by formula (II): 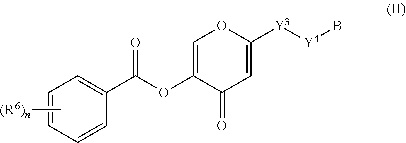
wherein:
B is selected from the group consisting of phenyl, heterocycloalkyl, and 5- or 6-membered heteroaryl, wherein each phenyl, heterocycloalkyl, and heteroaryl is independently optionally substituted with from one to three R²⁰;
Y³ and Y⁴ are each independently selected from the group consisting of a bond, —S—, —O—, —N(R¹)—, —C(R²)(R³)—, —C(O)— and —S(O)₂—;
n is 1, 2, or 3;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R⁶ is independently selected from the group consisting of halogen, —CN, —NO₂, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof;
provided that:
(A) when Y³ is —CH₂—; Y⁴ is —S—; and B is unsubstituted pyrimidin-2-yl; then A is not phenyl, 3-NO₂-4-Me-phenyl, 3-NO₂-phenyl, 4-Cl-phenyl, 4-NO₂-phenyl, 4-CF₃-phenyl, 4-Br-phenyl, 4-Me-phenyl, 4-OMe-phenyl, 4-OEt-phenyl, 4-OiPr-phenyl, 4-OBu-phenyl, 4-tBu-phenyl, 4-F-phenyl, 4-SO₂NMe₂-phenyl, 4-SO₂N(CH₂)₄-phenyl, 3,4-diMe-phenyl, 2-NO₂-5-Cl-phenyl, 2-Cl-5-NO₂-phenyl, 2-Me-3-NO₂-phenyl, 4-Ph-phenyl, 4-SO₂NEt₂-phenyl, 4-SO₂N(CH₂)₅-phenyl, 4-SO₂-morpholine-phenyl, or 2-NO₂-4,5-diOMe-phenyl;
(B) when Y³ is —CH₂—; Y⁴ is —S—; and B is 4-methyl-pyrimidin-2-yl; then A is not 3-NO₂-4-Cl-phenyl, 4-SO₂N(CH₂)₄-phenyl, 2-NO₂-5-Cl-phenyl, or 2-Cl-5-NO₂-phenyl; and
(C) when Y³ is —CH₂—; Y⁴ is —S—; and B is 4,6-dimethyl-pyrimidin-2-yl; then A is not 3-NO₂-4-Cl-phenyl, 3-NO₂-4-Me-phenyl, 4-SO₂NMe₂-phenyl, 2-NO₂-5-Cl-phenyl, 2-Cl-5-NO₂-phenyl, or 2-Me-3-NO₂-phenyl.

3. The method of claim 2, wherein —Y³—Y⁴— is —CH₂—, —CH₂—N(CH₂—C₆H₅)—, —CH₂—S—, or —CH₂—S(O)₂—.

4. The method of claim 2, wherein B is selected from the group consisting of phenyl, pyrimidinyl, morpholinyl, thiomorpholinyl, and piperazinyl, wherein each phenyl, pyrimidinyl, morpholinyl, thiomorpholinyl, and piperazinyl is optionally substituted with from one to three R²⁰.

5. The method of claim 2, wherein each R²⁰ is independently selected from the group consisting of halogen, —NO₂, —CN, —S(O)₂—N(CH₃)₂, —S(O)₂-heterocycloalkyl, alkyl, alkoxy, haloalkyl, haloalkoxy, phenyl, and heteroaryl.

6. The method of claim 1, wherein the compound of formula (I) is represented by formula (III): 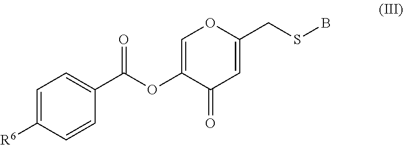
wherein:
B is selected from the group consisting of phenyl, heterocycloalkyl, and 5- or 6-membered heteroaryl, wherein each phenyl, heterocycloalkyl, and heteroaryl is independently optionally substituted with from one to three R²⁰;
R⁶ is selected from the group consisting of halogen, —CN, —NO₂, alkyl, cycloalkyl, heterocycloalkyl, alkoxy, haloalkyl, haloalkoxy, and 5- or 6-membered heteroaryl; and
each R²⁰ is independently selected from the group consisting of halogen, oxo, —C(O)-phenyl, alkyl, alkoxy, haloalkoxy, and phenyl;
or a pharmaceutically acceptable salt, polymorph, solvate, tautomer, or N-oxide thereof;
provided that when B is unsubstituted pyrimidin-2-yl; then R⁶ is not —F, —Cl, —Br, —NO₂, —CF₃, -Me, —OMe, —OEt, -OiPr, —OBu, or -tBu.

7. The method of claim 1, wherein the compound is selected from the group consisting of: 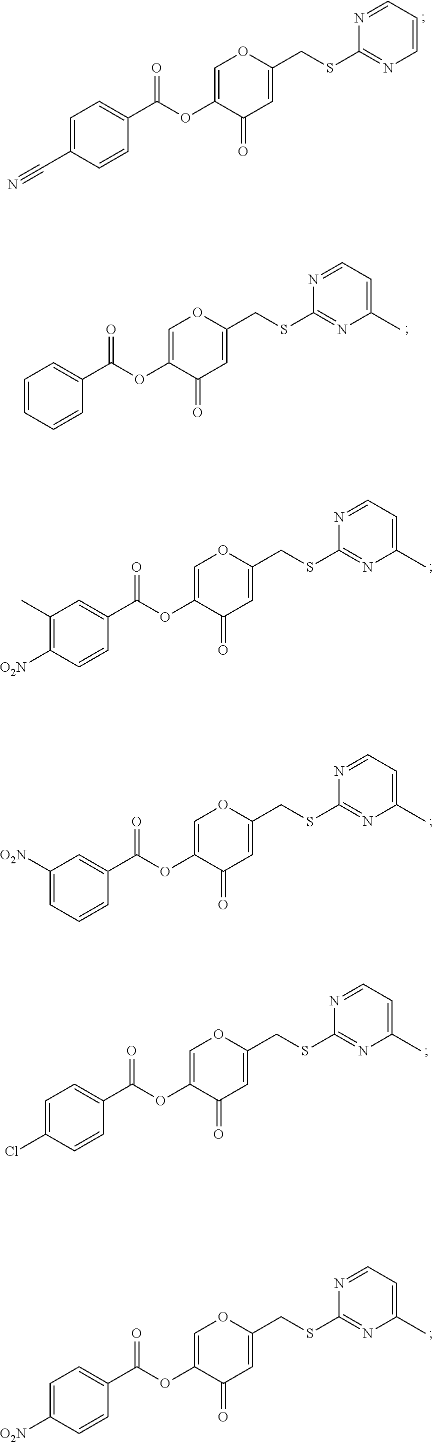 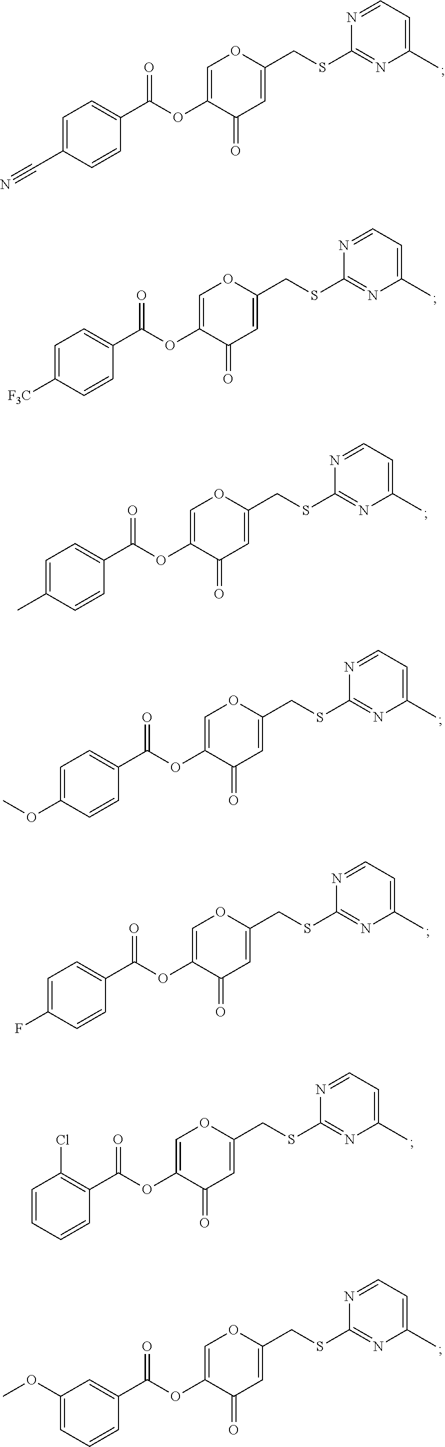 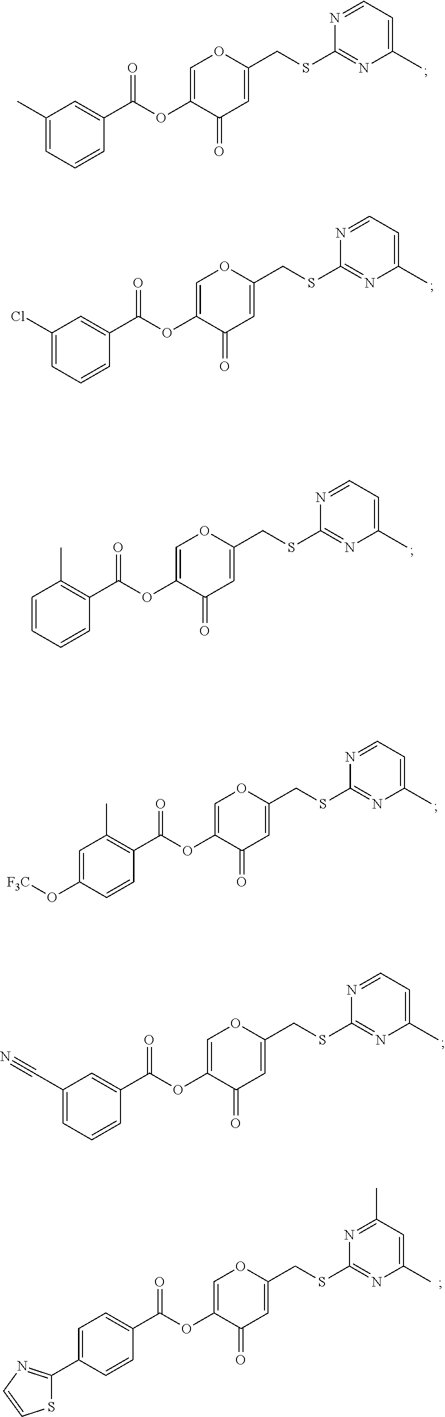 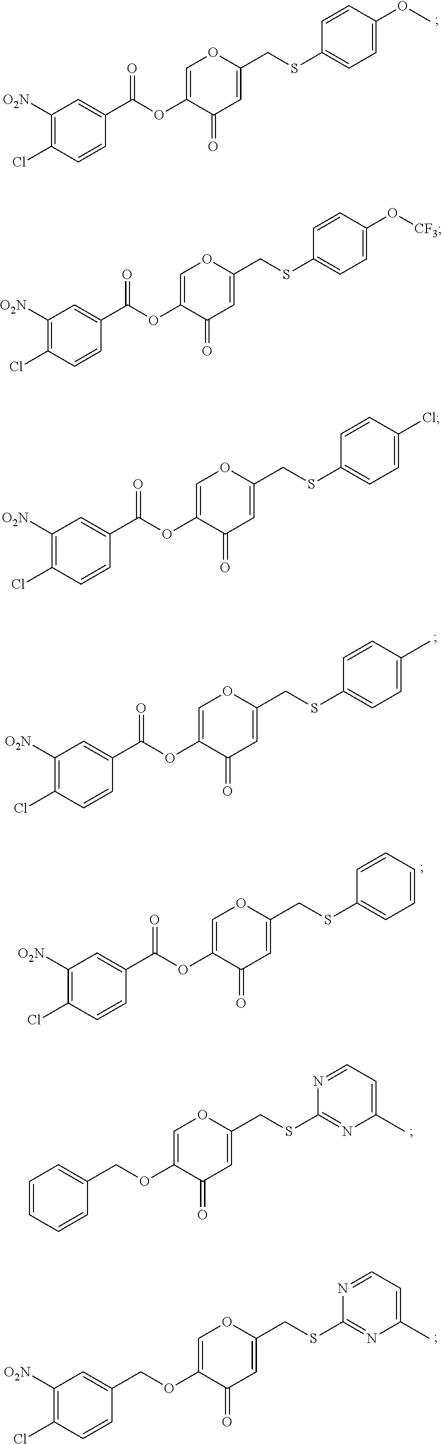 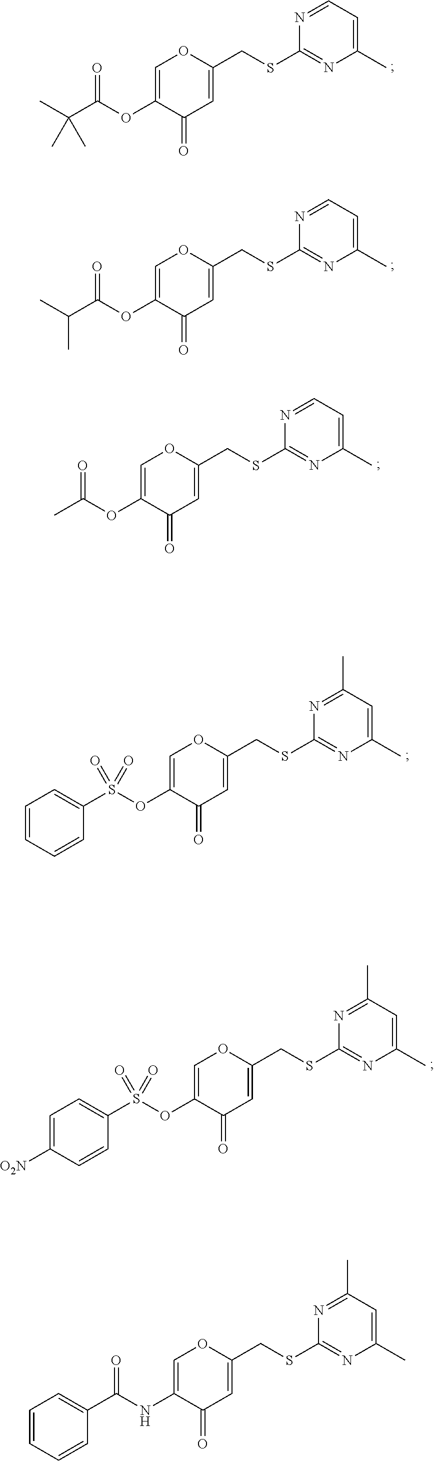 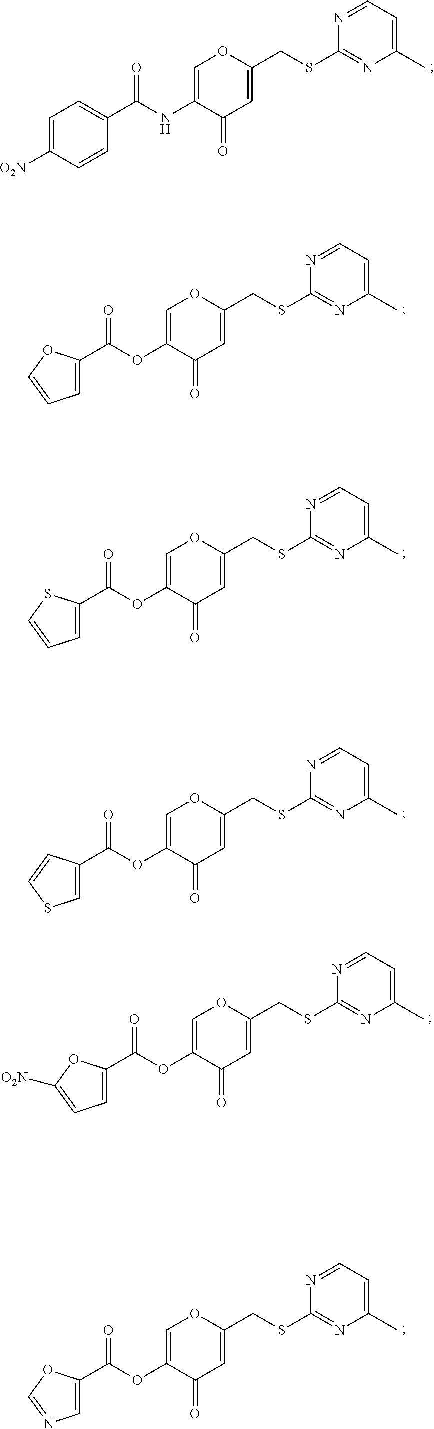 [SMILES: Cc1ccnc(SCc2cc(=O)c(NC(=O)c3ccc([N+](=O)[O-])cc3)co2)n1 ; Cc1ccnc(SCc2cc(=O)c(OC(=O)c3ccco3)co2)n1 ; Cc1ccnc(SCc2cc(=O)c(OC(=O)c3cccs3)co2)n1 ; Cc1ccnc(SCc2cc(=O)c(OC(=O)c3ccsc3)co2)n1 ; Cc1ccnc(SCc2cc(=O)c(OC(=O)c3ccc([N+](=O)[O-])o3)co2)n1 ; Cc1ccnc(SCc2cc(=O)c(OC(=O)c3cnco3)co2)n1] 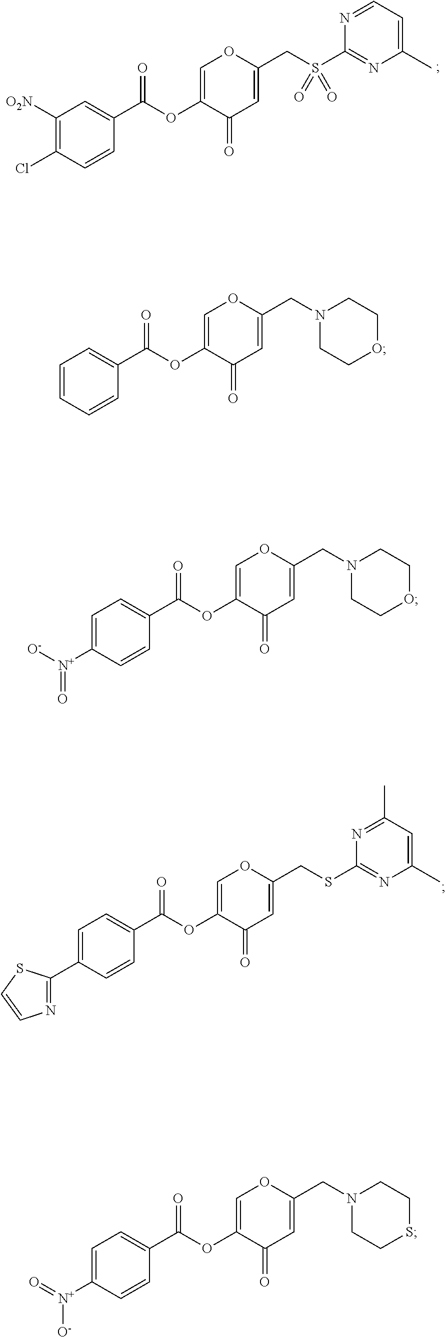 [SMILES: Cc1ccnc(S(=O)(=O)Cc2cc(=O)c(OC(=O)c3ccc(Cl)c([N+](=O)[O-])c3)co2)n1 ; O=C(Oc1coc(CN2CCOCC2)cc1=O)c1ccccc1 ; O=C(Oc1coc(CN2CCOCC2)cc1=O)c1ccc([N+](=O)[O-])cc1 ; Cc1cc(C)nc(SCc2cc(=O)c(OC(=O)c3ccc(-c4nccs4)cc3)co2)n1 ; O=C(Oc1coc(CN2CCSCC2)cc1=O)c1ccc([N+](=O)[O-])cc1] 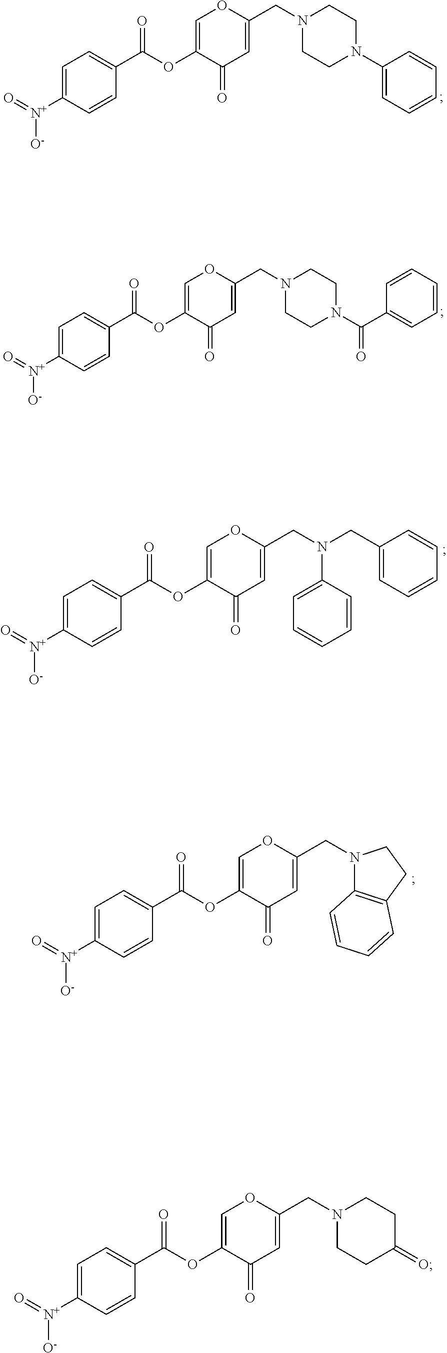 [SMILES: O=C(Oc1coc(CN2CCN(c3ccccc3)CC2)cc1=O)c1ccc([N+](=O)[O-])cc1 ; O=C(Oc1coc(CN2CCN(C(=O)c3ccccc3)CC2)cc1=O)c1ccc([N+](=O)[O-])cc1 ; O=C(Oc1coc(CN(Cc2ccccc2)c2ccccc2)cc1=O)c1ccc([N+](=O)[O-])cc1 ; O=C(Oc1coc(CN2CCc3ccccc32)cc1=O)c1ccc([N+](=O)[O-])cc1 ; O=C1CCN(Cc2cc(=O)c(OC(=O)c3ccc([N+](=O)[O-])cc3)co2)CC1] 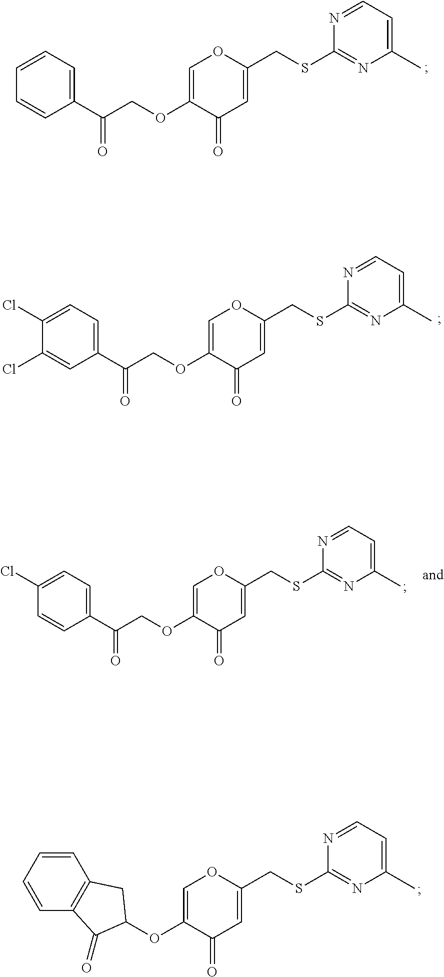 [SMILES: Cc1ccnc(SCc2cc(=O)c(OCC(=O)c3ccccc3)co2)n1 ; Cc1ccnc(SCc2cc(=O)c(OCC(=O)c3ccc(Cl)c(Cl)c3)co2)n1 ; Cc1ccnc(SCc2cc(=O)c(OCC(=O)c3ccc(Cl)cc3)co2)n1 ; Cc1ccnc(SCc2cc(=O)c(OC3Cc4ccccc4C3=O)co2)n1]
or a pharmaceutically acceptable salt, polymorph, solvate, tautomer, or N-oxide thereof.

8. A method of treating a disease mediated by apelin in a patient in need thereof, comprising administering to the patient therapeutically effective amount of a compound of formula (I): 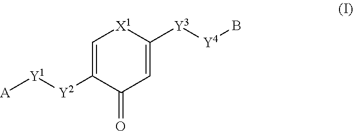
wherein:
X¹ is selected from the group consisting of —O— and —N(R⁵)—;
A is selected from the group consisting of alkyl, phenyl and 5- or 6-membered heteroaryl, wherein each phenyl and heteroaryl is independently optionally substituted with from one to three R²⁰;
B is selected from the group consisting of phenyl and 5- or 6-membered heteroaryl, wherein each phenyl and heteroaryl is independently optionally substituted with from one to three R²⁰;
Y¹ and Y² are each independently selected from the group consisting of a bond, —C(O)O—, —C(O)—, —CH₂O—, —N(R¹)—, —C(R²)(R³)— and —S(O)₂—;
Y³ and Y⁴ are each independently selected from the group consisting of a bond, —S—, —O—, —N(R¹)—, —C(R²)(R³)—, —C(O)— and —S(O)₂—;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof.

9. The method of claim 8, wherein the disease is selected from the group consisting of neoplasia, cardiovascular disease, peripheral vascular disease, hypertension, preeclampsia syndrome, abnormal angiogenesis, diabetes, ocular degeneration, idiopathic pulmonary fibrosis, would healing, chronic obstructive pulmonary disease, inflammatory disease such as arthritis, and inflammatory bowel disease, cardiovascular disease, avascular or ischemic insult, myocardial infarction, stroke, vaculitis, systemic or vascular sclerosis, gangrene, congelation, alopecia, eczema, ulcers, lymphedema, vascular hyperplasia, hemangioma, diabetic induce retinopathy, macular degenerative disease, psoriasis, or endometriosis.

10. The method of claim 8, wherein the disease is ocular degeneration.

11. The method of claim 8, wherein the disease is caused by abnormal angiogenesis.

12. The method of claim 11, wherein the disease is tumor.

13. The method of claim 12, wherein administration of the compound modifies tumor cell growth or endothelial cell growth in the patient, thereby treating the tumor or disease caused by abnormal angiogenesis.

14. A compound of formula (II): 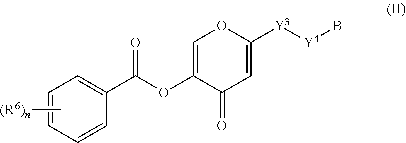
wherein:
B is selected from the group consisting of phenyl, heterocycloalkyl, and 5- or 6-membered heteroaryl, wherein each phenyl, heterocycloalkyl, and heteroaryl is independently optionally substituted with from one to three R²⁰;
Y³ and Y⁴ are each independently selected from the group consisting of a bond, —S—, —O—, —N(R¹)—, —C(R²)(R³)—, —C(O)— and —S(O)₂—;
n is 1, 2, or 3;
each R¹ is independently selected from the group consisting of hydrogen, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R² and R³ is independently selected from the group consisting of hydrogen, halogen, —CN, —N(R⁴)(R⁵), alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and heteroaryl is independently optionally substituted with from one to three R²¹;
each R⁴ and R⁵ is independently selected from the group consisting of hydrogen, alkyl, cycloalkyl, heterocycloalkyl, alkoxy, haloalkyl, haloalkoxy, phenyl, and heteroaryl; or R⁴ and R⁵ together with the nitrogen atom to which they are attached form a heterocycloalkyl or heteroaryl; and wherein each alkyl, cycloalkyl, heterocycloalkyl, alkoxy, haloalkyl, haloalkoxy, phenyl, and heteroaryl is independently optionally substituted with from one to three R²¹;
each R⁶ is independently selected from the group consisting of halogen, —CN, —NO₂, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹;
each R²⁰ is independently selected from the group consisting of halogen, —NO₂, —CN, oxo, —N(R⁴)(R⁵), —C(O)—N(R⁴)(R⁵), —C(O)-alkyl, —C(O)-cycloalkyl, —C(O)-heterocycloalkyl, —C(O)-phenyl, —C(O)-heteroaryl, —C(O)O-alkyl, —C(O)β-cycloalkyl, —C(O)O-heterocycloalkyl, —C(O)O-phenyl, —C(O)O-heteroaryl, —S(O)₂—N(R⁴)(R⁵), —S(O)₂-alkyl, —S(O)₂-cycloalkyl, —S(O)₂-heterocycloalkyl, —S(O)₂-phenyl, —S(O)₂-heteroaryl, alkyl, cycloalkyl, heterocycloalkyl, alkoxy, haloalkyl, haloalkoxy, phenyl, and 5- or 6-membered heteroaryl; wherein each alkyl, cycloalkyl, heterocycloalkyl, alkoxy, haloalkyl, haloalkoxy, phenyl, and 5- or 6-membered heteroaryl is independently optionally substituted with from one to three R²¹; and
each R²¹ is independently selected from the group consisting of halogen, —CN, —NH₂, —NO₂, —C(O)—NH₂, —C(O)-alkyl, —C(O)—O-alkyl, alkyl, alkoxy, haloalkyl, haloalkoxy and phenyl;
or a pharmaceutically acceptable salt, polymorph, solvate, tautomer, or N-oxide thereof;
provided that:
(A) when Y³ is —CH₂—; Y⁴ is —S—; and B is unsubstituted pyrimidin-2-yl; then A is not phenyl, 3-NO₂-4-Me-phenyl, 3-NO₂-phenyl, 4-Cl-phenyl, 4-NO₂-phenyl, 4-CF₃-phenyl, 4-Br-phenyl, 4-Me-phenyl, 4-OMe-phenyl, 4-OEt-phenyl, 4-OiPr-phenyl, 4-OBu-phenyl, 4-tBu-phenyl, 4-F-phenyl, 4-SO₂NMe₂-phenyl, 4-SO₂N(CH₂)₄-phenyl, 3,4-diMe-phenyl, 2-NO₂-5-Cl-phenyl, 2-Cl-5-NO₂-phenyl, 2-Me-3-NO₂-phenyl, 4-Ph-phenyl, 4-SO₂NEt₂-phenyl, 4-SO₂N(CH₂)₅-phenyl, 4-SO₂-morpholine-phenyl, or 2-NO₂-4,5-diOMe-phenyl;
(B) when Y³ is —CH₂—; Y⁴ is —S—; and B is 4-methyl-pyrimidin-2-yl; then A is not 3-NO₂-4-Cl-phenyl, 4-SO₂N(CH₂)₄-phenyl, 2-NO₂-5-Cl-phenyl, or 2-Cl-5-NO₂-phenyl; and
(C) when Y³ is —CH₂—; Y⁴ is —S—; and B is 4,6-dimethyl-pyrimidin-2-yl; then A is not 3-NO₂-4-Cl-phenyl, 3-NO₂-4-Me-phenyl, 4-SO₂NMe₂-phenyl, 2-NO₂-5-Cl-phenyl, 2-Cl-5-NO₂-phenyl, or 2-Me-3-NO₂-phenyl.

15. The compound of claim 14, wherein —Y³—Y⁴— is —CH₂—, —CH₂—N(CH₂—C₆H₅)—, —CH₂—S—, or —CH₂—S(O)₂—.

16. The compound of claim 14, wherein B is selected from the group consisting of phenyl, pyrimidinyl, morpholinyl, thiomorpholinyl, and piperazinyl, wherein each phenyl, pyrimidinyl, morpholinyl, thiomorpholinyl, and piperazinyl is optionally substituted with from one to three R²⁰.

17. The compound of claim 14, wherein each R²⁰ is independently selected from the group consisting of halogen, —NO₂, —CN, —S(O)₂—N(CH₃)₂, —S(O)₂-heterocycloalkyl, alkyl, alkoxy, haloalkyl, haloalkoxy, phenyl, and heteroaryl.

18. A compound of formula (III): 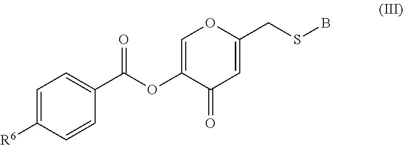
wherein:
B is selected from the group consisting of phenyl, heterocycloalkyl, and 5- or 6-membered heteroaryl, wherein each phenyl, heterocycloalkyl, and heteroaryl is independently optionally substituted with from one to three R²⁰;
R⁶ is selected from the group consisting of halogen, —CN, —NO₂, alkyl, cycloalkyl, heterocycloalkyl, alkoxy, haloalkyl, haloalkoxy, and 5- or 6-membered heteroaryl; and
each R²⁰ is independently selected from the group consisting of halogen, oxo, —C(O)-phenyl, alkyl, alkoxy, haloalkoxy, and phenyl;
or a pharmaceutically acceptable salt, polymorph, solvate, tautomer, or N-oxide thereof;
provided that when B is unsubstituted pyrimidin-2-yl; then R⁶ is not —F, —Cl, —Br, —NO₂, —CF₃, -Me, —OMe, —OEt, -OiPr, —OBu, or -tBu.

19. The compound of claim 14, wherein B is selected from the group consisting of:

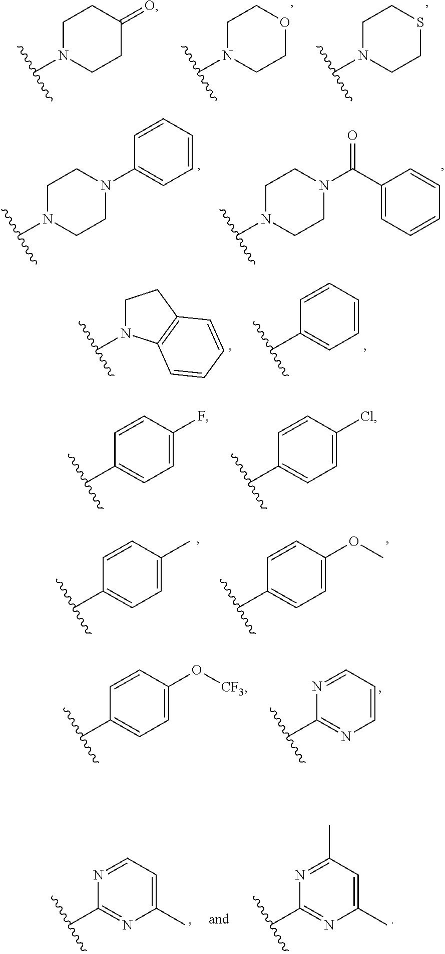

20. The compound of claim 14, wherein R⁶ is selected from the group consisting of bromo, —NO₂, —CN, or trifluoromethyl.

21. A compound selected from the group consisting of: 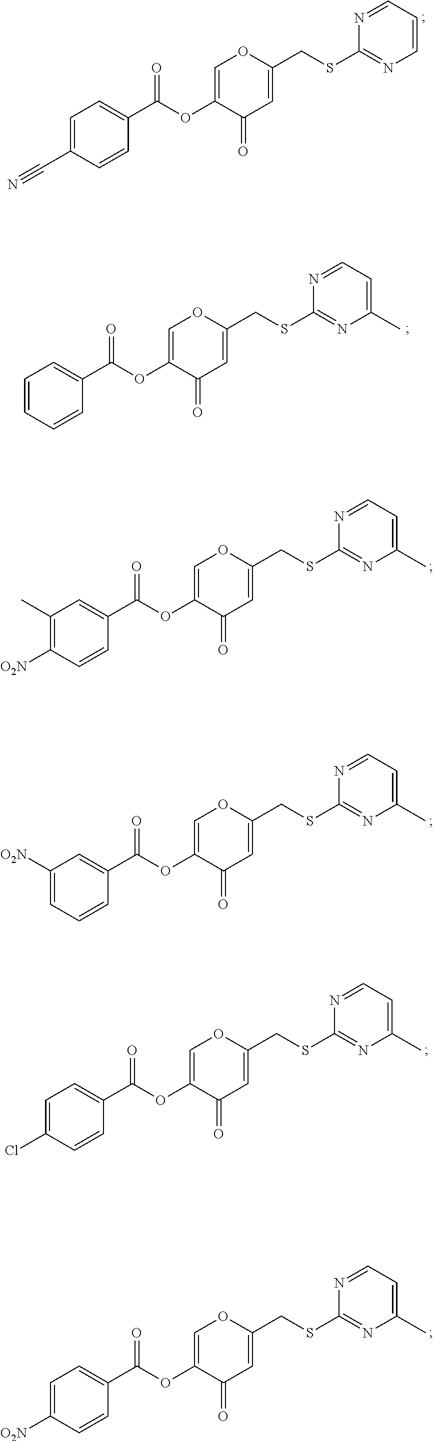 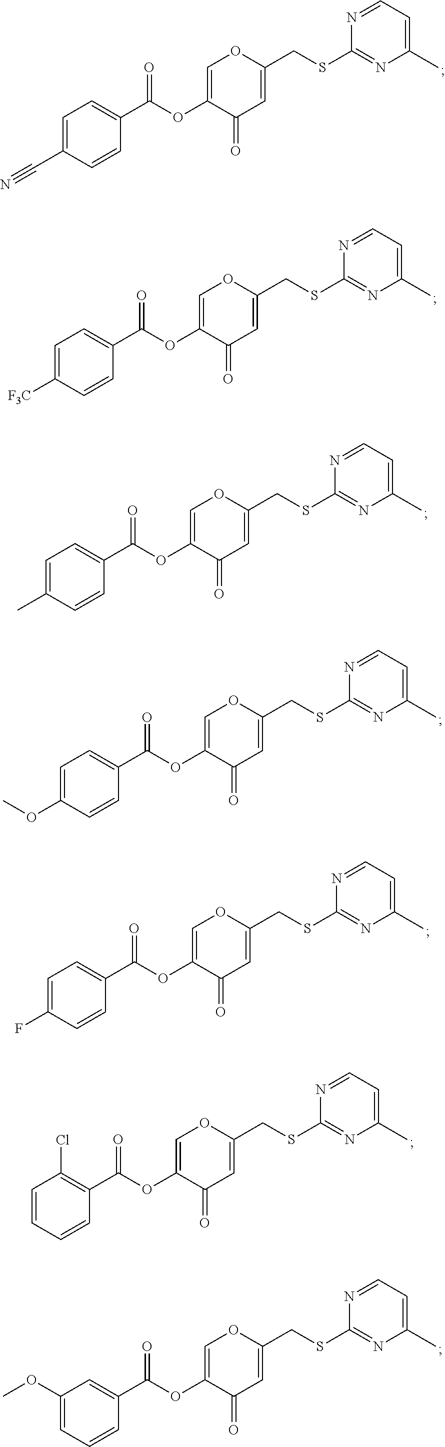 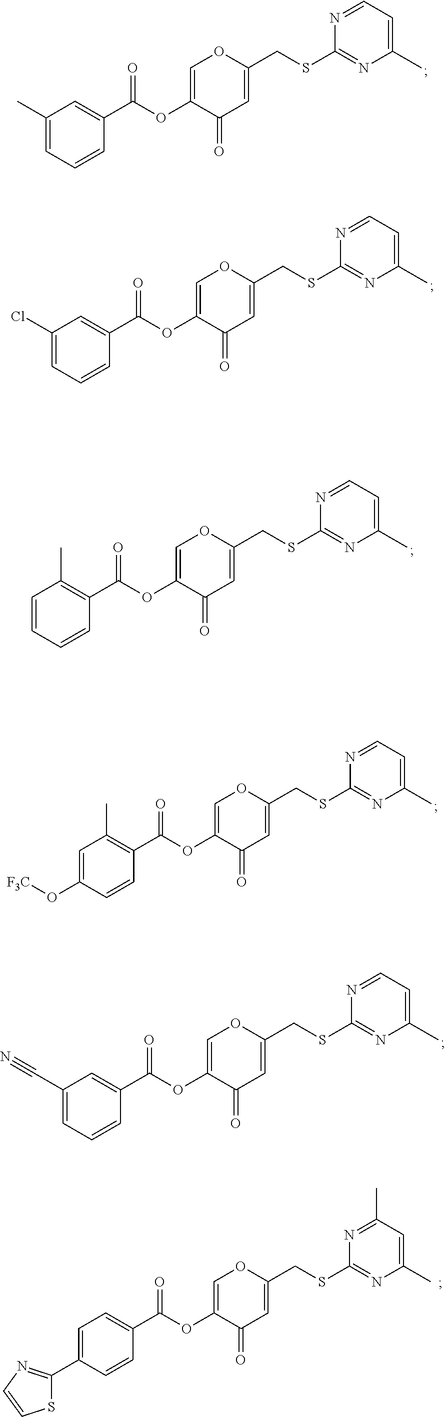 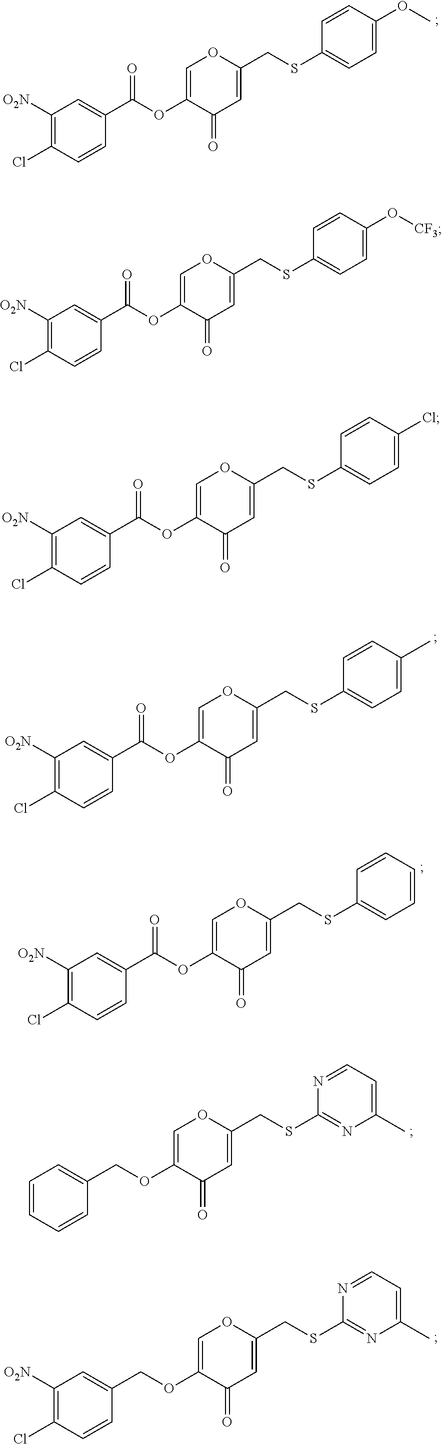 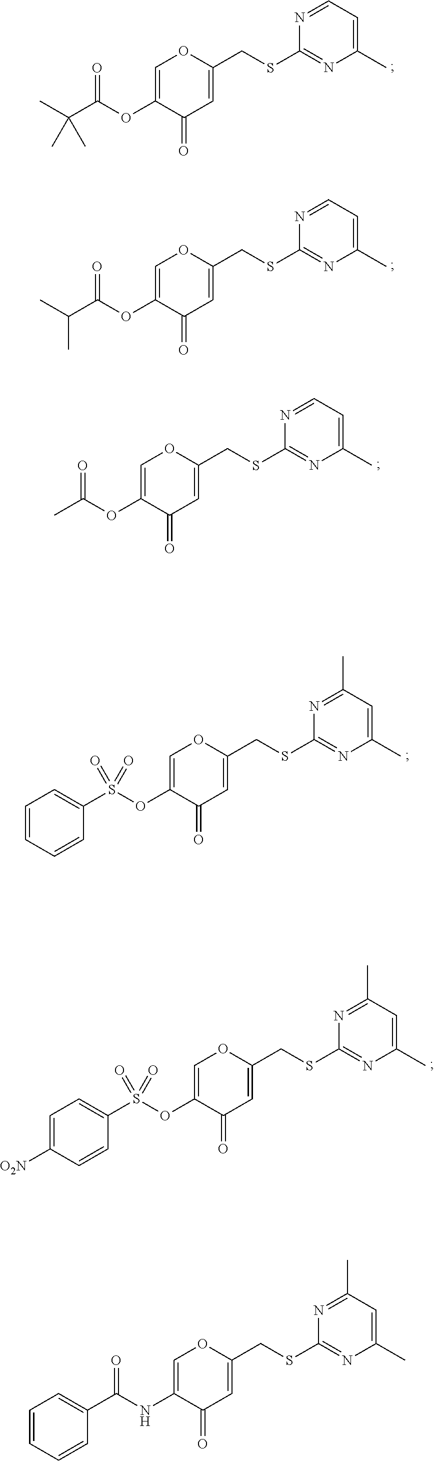 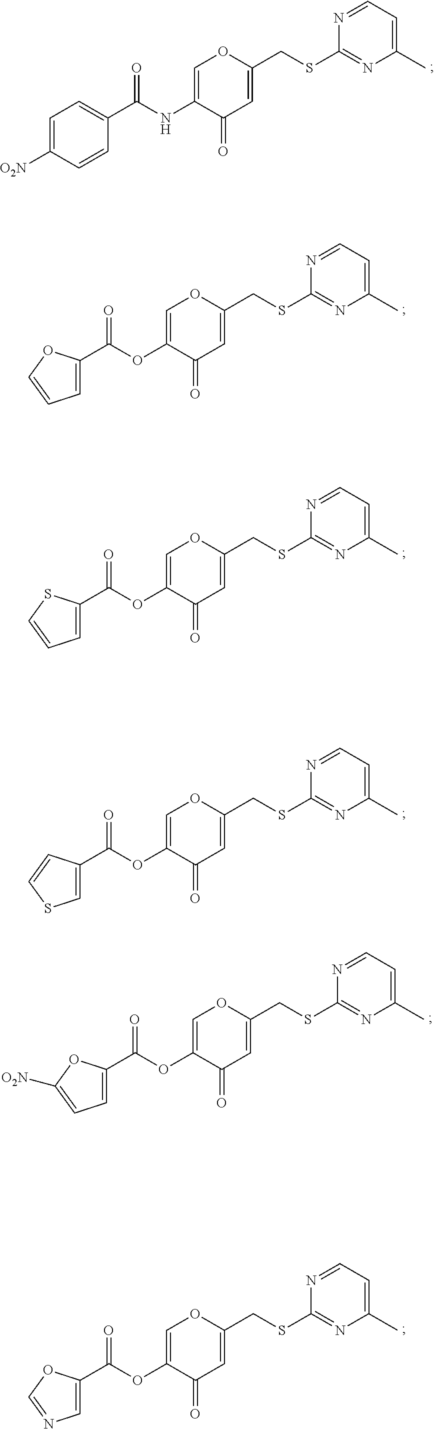 [SMILES: Cc1ccnc(SCc2cc(=O)c(NC(=O)c3ccc([N+](=O)[O-])cc3)co2)n1 ; Cc1ccnc(SCc2cc(=O)c(OC(=O)c3ccco3)co2)n1 ; Cc1ccnc(SCc2cc(=O)c(OC(=O)c3cccs3)co2)n1 ; Cc1ccnc(SCc2cc(=O)c(OC(=O)c3ccsc3)co2)n1 ; Cc1ccnc(SCc2cc(=O)c(OC(=O)c3ccc([N+](=O)[O-])o3)co2)n1 ; Cc1ccnc(SCc2cc(=O)c(OC(=O)c3cnco3)co2)n1] 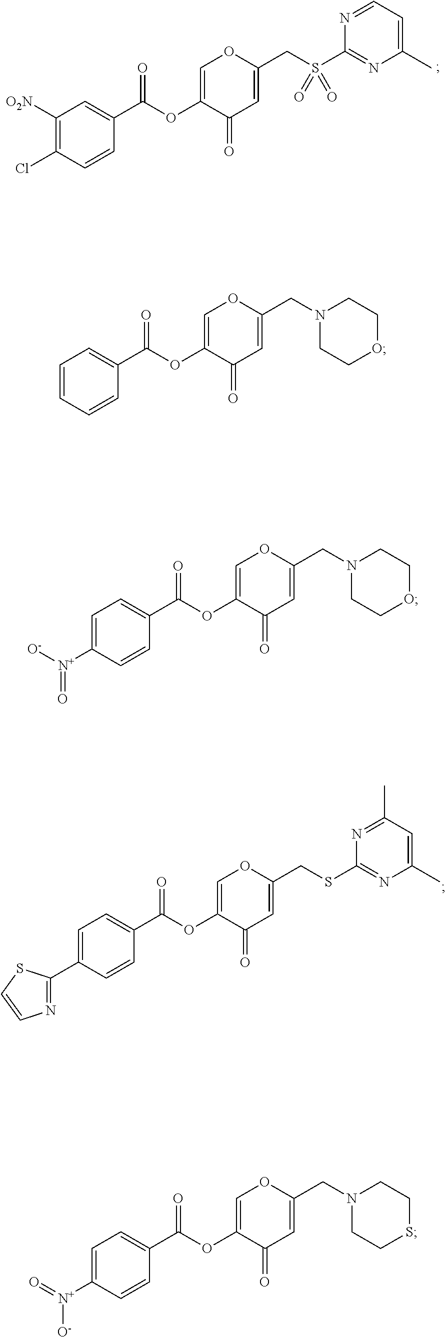 [SMILES: Cc1ccnc(S(=O)(=O)Cc2cc(=O)c(OC(=O)c3ccc(Cl)c([N+](=O)[O-])c3)co2)n1 ; O=C(Oc1coc(CN2CCOCC2)cc1=O)c1ccccc1 ; O=C(Oc1coc(CN2CCOCC2)cc1=O)c1ccc([N+](=O)[O-])cc1 ; Cc1cc(C)nc(SCc2cc(=O)c(OC(=O)c3ccc(-c4nccs4)cc3)co2)n1 ; O=C(Oc1coc(CN2CCSCC2)cc1=O)c1ccc([N+](=O)[O-])cc1] 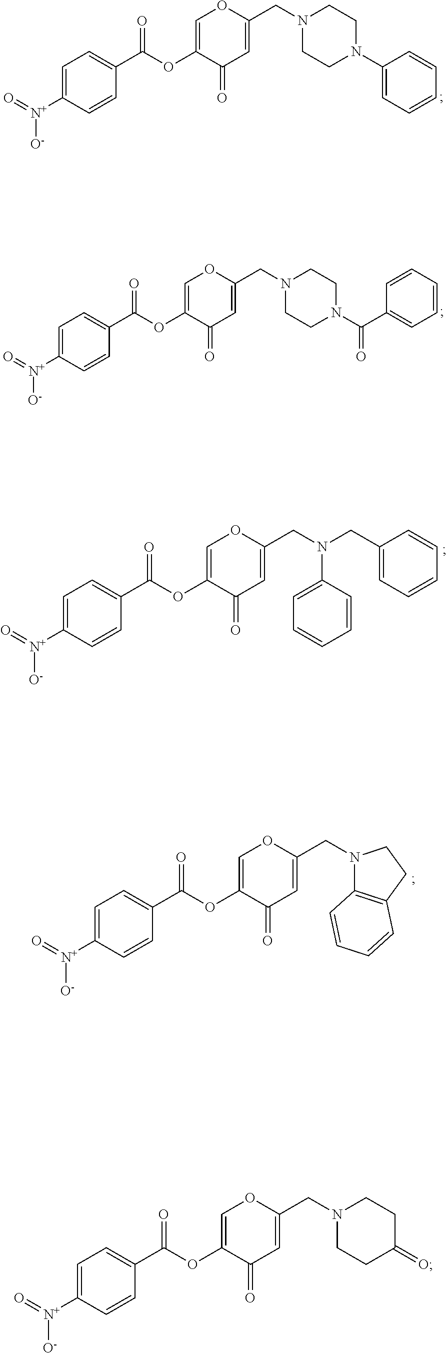 [SMILES: O=C(Oc1coc(CN2CCN(c3ccccc3)CC2)cc1=O)c1ccc([N+](=O)[O-])cc1 ; O=C(Oc1coc(CN2CCN(C(=O)c3ccccc3)CC2)cc1=O)c1ccc([N+](=O)[O-])cc1 ; O=C(Oc1coc(CN(Cc2ccccc2)c2ccccc2)cc1=O)c1ccc([N+](=O)[O-])cc1 ; O=C(Oc1coc(CN2CCc3ccccc32)cc1=O)c1ccc([N+](=O)[O-])cc1 ; O=C1CCN(Cc2cc(=O)c(OC(=O)c3ccc([N+](=O)[O-])cc3)co2)CC1] 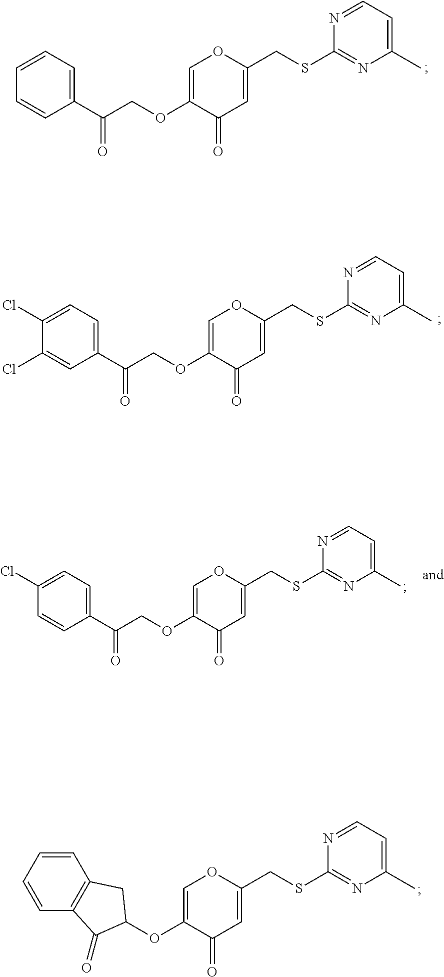 [SMILES: Cc1ccnc(SCc2cc(=O)c(OCC(=O)c3ccccc3)co2)n1 ; Cc1ccnc(SCc2cc(=O)c(OCC(=O)c3ccc(Cl)c(Cl)c3)co2)n1 ; Cc1ccnc(SCc2cc(=O)c(OCC(=O)c3ccc(Cl)cc3)co2)n1 ; Cc1ccnc(SCc2cc(=O)c(OC3Cc4ccccc4C3=O)co2)n1]
or a pharmaceutically acceptable salt, polymorph, solvate, tautomer, or N-oxide thereof.

22. A pharmaceutical composition comprising a compound of claim 14 or a pharmaceutically acceptable salt, polymorph, solvate, tautomer, or N-oxide thereof, and a pharmaceutically acceptable excipient.
